(19) United States
(12) Patent Application Publication
(10) Pub. No.: US 2004/0072796 A1
(43) Pub. Date: Apr. 15, 2004

(54) Method and composition for preventing pain in sickle cell patients

(76) Inventors: Stephen H. Embury, Half Moon Bay, CA (US); Neil M. Matsui, San Francisco, CA (US)

Correspondence Address: PERKINS COIE LLP, P.O. BOX 2168, MENLO PARK, CA, 94026, US

(21) Appl. No.: 10/418,653

(22) Filed: Apr. 18, 2003

Related U.S. Application Data
(60) Provisional application No. 60/373,841, filed on Apr. 18, 2002.
(60) Provisional application No. 60/373,842, filed on Apr. 18, 2002.
(60) Provisional application No. 60/373,844, filed on Apr. 18, 2002.

(51) Int. Cl. A61K 31/727

(52) U.S. Cl. 514/56

(57) ABSTRACT

A method of preventing pain in a sickle cell patient is disclosed. The method includes orally administering to the patient, an amount of an active agent effective on oral administration to inhibit binding of the patient's sickle erythrocytes to P-selectin on the patient's vascular endothelium. The inhibition may be evidenced in a number of ways. The active agent administration inhibits the adhesion of sickle erythrocytes to vascular endothelium in the patient, thereby preventing patient pain associated with vascular occlusion. Also disclosed are compositions useful in practicing the method.

This application claims the benefit of U.S. Provisional Application No. 60/373,841, filed Apr. 18, 2002; U.S. Provisional Application No. 60/373,842, filed Apr. 18, 2002; and U.S. Provisional Application No. 60/373,844, filed Apr. 18, 2002, each of which is incorporated herein by reference in its entirety.

### FIELD OF THE INVENTION

The present invention relates to compositions and methods for preventing or reducing pain in sickle cell patients due to vascular occlusion.

### BACKGROUND OF THE INVENTION

Sickle cell disease is a debilitating inherited disorder of red blood cells that is characterized by lifelong anemia, recurrent attacks of severe pain, failure of certain organs to function normally, and premature death. The inherited mutation responsible for sickle cell disease is a single base mutation in the gene that makes one of the two globin subunits of hemoglobin, the molecule within red blood cells that carries oxygen. The result of the mutation is a hemoglobin molecule (sickle hemoglobin; Hb S) that is poorly soluble when it lacks oxygen. The result of Hb S losing oxygen is that it comes out of solution, polymerizes, and turns the normally disc-shaped red blood cells into rigid, misshapen sickle cells.

Since the average red cell releases its oxygen approximately once a minute as it traverses into the small blood vessels of the circulation, this sickling is seen to be persistent and unrelenting. Yet, most of the sickle red blood cells have reentered the larger vessels in which the red cell rigidity is not harmful. It is believed that the pain and organ failure observed in sickle patients which result from blockage of blood flow in small vessels occurs when the transit of sickle red cells through small vessels is delayed. In this instance polymerization and sickling occur in small vessels which they can block.

Most patients with sickle cell disease can be expected to survive into adulthood, but still face a lifetime of crises and complications, including chronic hemolytic anemia, vaso-occlusive crises and pain, and the side effects of therapy. Currently, most common therapeutic interventions include blood transfusions, opioid and hydroxyurea therapies (see, for example, S. K. Ballas in Cleveland Clin. J. Med., 66:48-58 (1999). However, all of these therapies are associated with some undesirable side-effects. For example, repeated blood transfusions are known to be associated with the risks of transmission of infectious disease, iron overload, and allergic and febrile reactions. Complications of opioid therapy may include addiction, seizures, dependency, respiratory depression and constipation.

Hydroxyurea, an inhibitor of ribonucleotide reductase, acts by impairing DNA synthesis in cells (see, for example, J. W., Yarbro in Semin. Oncol., 19:1-10 (1992). For decades, hydroxyurea has been used clinically as an anti-cancer agent for the treatment of leukemia, skin and other cancers. Since early 1980, hydroxyurea has been used to treat patients with sickle cell disease. Sickle cell patients treated with hydroxyurea often seem to have fewer painful crises of vaso-occlusion, fewer hospitalizations and fewer episodes of acute chest syndrome (See, for example, S. Charache et al. in New Engl. J. Med., 332:1317-1322 (1995); S. Charache et al. in Med., 75:300-326 (1996); and J. L. Bauman et al. in Arch. Intern Med., 141:260-261 (1981)). It appears that hydroxyurea treatment increases fetal hemoglobin levels in the red cell, which in turn inhibits the aggregation of sickle cell hemoglobin. However, not all patients in these studies benefited from hydroxyurea treatment, and painful crises of vaso-occlusion were not eliminated in most patients. In fact, a recent clinical trial showed that after a 2-year treatment, fetal hemoglobin levels of patients assigned to the hydroxyurea arm of the study did not differ markedly from their pretreatment levels (see, for example, S. Charache in Seminars in Hematol., 34:15-21 (1997)). Thus, the mechanism of action of hydroxyurea in the treatment of sickle cell anemia remains unclear.

In addition to the limited effectiveness of hydroxyurea therapy, such treatment causes a wide range of undesirable side-effects. The primary side-effect of hydroxyurea is myelosuppression (neutropenia and thrombocytopenia), placing patients at risks for infection and bleeding. In addition, long-term treatment with hydroxyurea may cause a wide spectrum of diseases and conditions, including multiple skin tumors and ulcerations, fever, hepatitis, hyperpigmentation, scaling, partial alopecia, atrophy of the skin and subcutaneous tissues, nail changes and acute interstitial lung disease (see, for example, P. J. M. Best et al. in Mayo Clin. Proc., 73:961-963 (1998); M. S. Kavuru et al. in Cerebral Arterial Thrombosis, 87:767-769 (1994); M. J. F. Starmans-Kool et al. in Ann. Hematol, 70:279-280 (1995); and M. Papi et al. in Am Acad. Dermatol., 28:485-486 (1993)).

Because sickle cell disease is a genetic disease, in theory, a gene therapy approach should be considered. In fact, gene therapies employing either ribozyme-mediated or retroviral vector-mediated approaches to replacing the defective human β-globin gene are being actively developed for the treatment of sickle cell disease (see, for example, D. J. Weatherall, Curr. Biol., 8:R696-8 (1998); and R. Pawliuk et al., Ann. N.Y. Acad. Sci., 850:151-162 (1998)). However, the gene therapy approach to treating sickle cell disease involves bone marrow transplantation, a procedure which has its own inherent toxicities and risks (for a review, see, C. A. Hillery in Curr. Opin. Hematol., 5:151-5 (1998)). Accordingly, there is still a need in the art for new methods that are useful in treating sickle cell anemia or one or more of the symptoms associated with sickle cell disease.

Pain is a major factor in sickle cell patients, particularly those with severe manifestations of the disease. The pain is sufficiently debilitating to interfere with a normal life style, and can even be so severe as to require hospitalization. Although methods for the treatment or prevention of pain in sickle cell patients have been proposed, these are either relatively ineffective and/or require administration by injection. It would therefore be highly desirable to provide an effective therapeutic for sickle cell patients that can be administered orally. The present invention is designed to meet these needs.

### SUMMARY OF THE INVENTION

Accordingly, it is an object of the invention to provide a method of enhancing blood flow and/or preventing pain in a sickle cell patient. The method includes orally administering to the patient, an amount of heparin effective on oral administration to inhibit binding of the patient's sickle erythrocytes to P-selectin on the patient's vascular endothelium. This inhibition is evidenced by one or more of (i) enhanced microvascular blood flow in conjunctivae of the patient relative to microvascular blood flow prior to treatment, (ii) enhanced vascular endothelial well-being in the patient relative to vascular endothelial well-being prior to treatment, and/or (iii) prevention or reduced frequency of pain crises in the patient relative to pain crises prior to treatment. Administration of heparin inhibits the adhesion of sickle erythrocytes to vascular endothelium in the patient, thereby preventing patient pain associated with vascular occlusion.

In one embodiment of the invention, the inhibition is evidenced by enhanced microvascular blood flow in conjunctivae of the patient relative to microvascular blood flow prior to treatment. Preferably, the blood flow is monitored with computer-assisted intravital microscopy and/or Laser-Doppler velocimetry in vivo.

In another embodiment, the inhibition is evidenced by enhanced vascular endothelial well-being in the patient relative to vascular endothelial well-being prior to treatment as determined by one or more surrogate markers of vascular endothelial well-being. The surrogate marker may be one or more of: soluble P-selectin (sP-sel), vascular endothelial cell adhesion molecule-1 (sVCAM-1), tumor necrosis factor-a (TNFa), Interleukin-1b (IL-1b), IL-6, IL-8, IL-10, a2-macroglobulin, C-reactive protein (CRP), high sensitivity CRP, soluble interleukin-2 receptor (sIL-2R), substance P, endothelin-1, circulating endothelial cells (CEC), microparticles (MP) from the plasma membranes of endothelial cells, MP from monocytes, platelets, and sickle RBC.

In yet another embodiment, the inhibition is evidenced by prevention or reduction in frequency of pain crises in the patient relative to pain crises prior to treatment.

In one embodiment of the invention, the heparin administered is a non-anticoagulant form of heparin formed by desulfating heparin at the 2-O position of uronic acid residues and the 3-O position of glucosamine residues of heparin. In another embodiment, the heparin administered is unfractionated porcine heparin. In yet another embodiment, the heparin administered is produced by treating porcine heparin with a mixture of heparinases, under conditions effective to produce an average molecular weight of heparin between 4 and 6 kilodaltons.

In a preferred embodiment, the heparin administered is complexed with an enhancer compound effective to enhance the uptake of the heparin from the gastrointestinal (GI) tract into the bloodstream. The enhancer compound may be selected from the group consisting of sodium N-[8-(2-hydroxybenzoyl)amino] caprylate (SNAC), sodium N-[8-(2-hydroxybenzoyl)amino] decanoate (SNAD), Orasomes, Promdas, Locdas, Hydroance, Lipral, Labrasol (caprylocaproyl macrogolglycerides), D-α-tocopheryl polyethylene glycol 1000 succinate (TPGS), DOCA, alginate/poly-L-lysine microparticles, polycarbophil, hydroxypropyl methylcellulose, carbopol 934, sodium salicylate, polyoxyethylene-9-lauryl ether, poly(ethylcyanoacrylate) (PECA), 2-alkoxy-3-alkylamidopropylphosphocholines, dodecylphosphocholine (DPC), and poly(diethyl)methylidenemalonate (DEMM). Preferably, the enhancer compound is Hydroance.

In one embodiment of the invention, the heparin administered is in a tablet or capsule designed to release heparin after the heparin has entered the intestine. In another embodiment, the administering is carried out on a daily basis, at a daily dose of between about 40 mgs to about 2700 mgs heparin. Preferably, daily dose is between about 50 mgs to about 600 mgs heparin.

In another aspect of the invention, a composition for use in preventing pain in a sickle-cell patient is provided. The composition includes heparin contained in a solid or capsule form suitable for oral administration, at a total dose of between about 50 to 500 mg heparin.

In one embodiment, the heparin is a non-anticoagulant form of heparin formed by desulfating heparin at the 2-O position of uronic acid residues and the 3-O position of glucosamine residues of heparin. Alternatively, the heparin is unfractionated porcine heparin. In another embodiement, the heparin is produced by treating porcine heparin with a mixture of heparinases, under conditions effective to produce an average molecular weight of heparin between 4 and 6 kilodaltons.

Preferably, the heparin is complexed with an enhancer compound effective to enhance the uptake of the heparin from the GI tract into the bloodstream. The enhancer may be selected from the group consisting of sodium N-[8-(2-hydroxybenzoyl)amino] caprylate (SNAC), sodium N-[8-(2-hydroxybenzoyl)amino] decanoate (SNAD), Orasomes, Promdas, Locdas, Hydroance, Lipral, Labrasol (caprylocaproyl macrogolglycerides), D-α-tocopheryl polyethylene glycol 1000 succinate (TPGS), DOCA, alginate/poly-L-lysine microparticles, polycarbophil, hydroxypropyl methylcellulose, carbopol 934, sodium salicylate, polyoxyethylene-9-lauryl ether, poly(ethylcyanoacrylate) (PECA), 2-alkoxy-3-alkylamidopropylphosphocholines, dodecylphosphocholine (DPC), and poly(diethyl)methylidenemalonate (DEMM). Preferably, the enhancer compound is Hydroance.

It is another object of the invention to provide a method of preventing pain in a sickle-cell patient that includes administering to the patient, an agent selected from the group consisting of monoclonal antibodies directed against P-selectin or its ligand PSGL-1; heparinoids that block P-selectin binding; the carbohydrate molecule fucoidin and synthetic sugar derivatives such as OJ-R9188 which block selectin-ligand interactions; the carbon-fucosylated derivative of glycyrrhetinic acid GM2296 and other sialyl Lewis X glycomimetic compounds; inhibitors of P-selectin expression such as mycophenolate mofetil, the proteasome inhibitor ALLN, and antioxidants such as PDTC; sulfatide and sulfatide analogues such as BMS-190394; the 19 amino acid terminal peptide of PSGL-1, other PSGL-1 peptides, PSGL-1 fusion proteins, PSGL-1 analogues, and selective inhibitors of PSGL-1 binding such as beta-C-mannosides; benzothiazole compounds derived from ZZZ21322 such as Compound 2; and statins such as Simvastatin.

The agent is administered in an amount effective to inhibit in the binding of sickle erythrocytes to P-selectin on the vascular endothelium. This inhibition is evidenced by one or more of (i) enhanced microvascular blood flow in conjunctivae of the patient relative to microvascular blood flow prior to treatment, (ii) enhanced vascular endothelial well-being in the patient relative to vascular endothelial well-being prior to treatment, and/or (iii) prevention or reduced frequency of pain crises in the patient relative to pain crises prior to treatment. This administration inhibits the adhesion of sickle erythrocytes to vascular endothelium in the patient, thereby preventing patient pain associated with vascular occlusion.

Preferably the agent is administered orally. The agent may be complexed with an enhancer compound effective to enhance the uptake of the agent from the GI tract into the bloodstream.

In one embodiment, the enhancer compound is selected from the group consisting of sodium N-[8-(2-hydroxybenzoyl)amino] caprylate (SNAC), sodium N-[8-(2-hydroxybenzoyl)amino] decanoate (SNAD), Orasomes, Promdas, Locdas, Hydroance, Lipral, Labrasol (caprylocaproyl macrogolglycerides), D-α-tocopheryl polyethylene glycol 1000 succinate (TPGS), DOCA, alginate/poly-L-lysine microparticles, polycarbophil, hydroxypropyl methylcellulose, carbopol 934, sodium salicylate, polyoxyethylene-9-lauryl ether, poly(ethylcyanoacrylate) (PECA), 2-alkoxy-3-alkylamidopropylphosphocholines, dodecylphosphocholine (DPC), and poly(diethyl)methylidenemalonate (DEMM). Preferably, the enhancer compound is Hydroance.

In another embodiment, the agent is administered is in a tablet or capsule protected form designed to increase absorption from the GI tract.

These and other objects and features of the invention will be more fully appreciated when the following detailed description of the invention is read in conjunction with the accompanying drawings.

### BRIEF DESCRIPTION OF THE DRAWINGS

FIGS. 1A-1E show the importance of P-selectin in the flow adhesion of sickle erythrocytes to thrombin-stimulated endothelium in vitro. The number of sickle cells adhering to HUVECs and the rolling velocities of the adhering cells are given. (A and B) In 10 experiments, the rolling adhesion of sickle cells to HUVECs was examined prior to and after treatment of HUVECs with thrombin. The rolling adhesion of sickle cells to thrombin-treated HUVECs was then examined in the presence of anti-P-selectin mAb 9E1. Statistically significant differences compared with untreated HUVECs and to thrombin-treated HUVECs are indicated. (C and D) In 3 experiments the rolling adhesion of sickle cells to thrombin-treated HUVECs also was examined in the presence of nonblocking anti-P-selectin mAb AC1.2. Statistically significant differences compared with untreated HUVECs and to thrombin-treated HUVECs are indicated. (E) In 10 experiments the firm adhesion of sickle cells to HUVECs was examined prior to and after treatment of HUVECs with thrombin. Statistically significant differences are indicated.

FIGS. 2A-2B show that sickle cells adhere to immobilized P-selectin under flow conditions in vitro. The number of sickle cells adhering to immobilized protein and the rolling velocities of the adhering cells were examined. In 5 experiments the rolling adhesion of sickle cells to BSA, immobilized recombinant Siglec, or immobilized recombinant P-selectin were examined. Statistically significant differences compared to immobilized BSA and to immobilized recombinant Siglec are indicated.

FIGS. 3A-3C show that heparin inhibits the flow adhesion of sickle erythrocytes to thrombin-stimulated endothelium in vitro. (A,B) The number of sickle cells adhering to HUVECs and the rolling velocities of the adhering cells were examined. In 6 experiments the rolling adhesion of sickle cells to thrombin-treated HUVECs was examined in the presence of anti-P-selectin mAb 9E1 or in the presence of unfractionated heparin. Statistically significant differences compared to thrombin-treated HUVECs are indicated. (C) In 3 experiments the firm adhesion of sickle cells to thrombin-treated HUVECs was examined in the presence of anti-P-selectin mAb 9E1 or in the presence of unfractionated heparin. Statistically significant differences are indicated.

FIGS. 4A-4B show that heparin inhibits the flow adhesion of sickle erythrocytes to immobilized P-selectin in vitro. The number of sickle cells adhering to immobilized protein and the rolling velocities of the adhering cells were examined. In 3 experiments the rolling adhesion of sickle cells to immobilized recombinant P-selectin was examined in the presence and absence of unfractionated heparin. Statistically significant differences compared with immobilized recombinant P-selectin are indicated.

FIGS. 5A-5B show that clinically obtainable concentrations of clinical-grade heparin inhibit the adhesion of sickle cells to P-selectin. The number of sickle cells adhering to immobilized protein (A) and the rolling velocities (B) of the adherent sickle cells on immobilized P-selectin were examined in the presence of 0.05, 0.5, 5, or 50 U/mL of laboratory-grade heparin (Sigma) or clinical-grade heparin (Clinical). Adherent sickle cells also were examined for number of cells rolling on BSA (B) or on immobilized P-selectin (P) and their velocities in the absence of heparin.

### DETAILED DESCRIPTION OF THE INVENTION

### I. Definitions

Unless otherwise indicated, all technical and scientific terms used herein have the same meaning as they would to one skilled in the art of the present invention.

Practitioners are particularly directed to Sickle Cell Disease: Basic Principles and Clinical Practice ((1994) NY Raven Press, Eds. Embury S. H., et al.). It is to be understood that this invention is not limited to the particular methodology, protocols, and reagents described, as these may vary. All publications and patents cited herein are expressly incorporated herein by reference for the purpose of describing and disclosing compositions and methodologies which might be used in connection with the invention.

The term “heparin” refers to heparin, low molecular weight heparin, unfractionated heparin, desulfated heparin at the 2-O position of uronic acid residues and/or the 3-O position of glucosamine residues of heparin, heparan, heparin and heparan salts formed with metallic cations (e.g., sodium, calcium or magnesium, preferably sodium) or organic bases (e.g., diethylamine, triethylamine, triethanolamine, etc.), heparin and heparan esters, heparin and heparan fatty acid conjugates, heparin and heparan bile acid conjugates, heparin sulfate, and heparan sulfate.

The terms “active agent,” “drug” and “pharmacologically active agent” are used interchangeably herein to refer to a chemical material or compound which, when administered to an organism (human or animal, generally human) induces a desired pharmacologic effect. In the context of a preferable embodiment of the present invention, the terms refer to a compound that is capable of being delivered orally.

The term “enhancer” is used herein to refer to compounds that disrupt or modify the absorptive surface of a targeted site (such as wetting) to improve absorption across a membrane.

The term “vascular endothelium” refers to a thin layer of flat epithelial cells that lines, for example, blood vessels. The vascular endothelium plays important roles in the regulation of vascular tone, hemostasis, immune and inflammatory responses (see, e.g., Vane J., et al., (1990) New Engl. J. Med 323: 27-31.)

As used herein, the term “inhibit binding” relative to the effect of a given concentration of a particular active agent on the binding of a P-selectin to sickle erythrocytes refers to a decrease in the amount of binding of the P-selectin to sickle erythrocytes relative to the amount of binding in the absence of the same concentration of the particular active agent, and includes both a decrease in binding as well as a complete inhibition of binding.

By the terms “effective amount” or “pharmaceutically effective amount” of an agent as provided herein are meant a nontoxic but sufficient amount of the agent to provide the desired therapeutic effect. As will be pointed out below, the exact amount required will vary from subject to subject, depending on age, general condition of the subject, the severity of the sickle cell condition, and the particular active agent administered, and the like. An appropriate “effective” amount in any individual case may be determined by one of ordinary skill in the art by reference to the pertinent texts and literature and/or using routine experimentation.

By “pharmaceutically acceptable” carrier is meant a carrier comprised of a material that is not biologically or otherwise undesirable. The term “carrier” is used generically herein to refer to any components present in the pharmaceutical formulations other than the active agent or agents, and thus includes diluents, binders, lubricants, disintegrants, fillers, coloring agents, wetting or emulsifying agents, pH buffering agents, preservatives, and the like.

Similarly, a “pharmaceutically acceptable” salt or a “pharmaceutically acceptable” derivative of a compound as provided herein is a salt or other derivative which is not biologically or otherwise undesirable.

The term “controlled release” is intended to refer to any drug-containing formulation in which the manner and profile of drug release from the formulation are controlled. The term “controlled release” refers to immediate as well as nonimmediate release formulations, with nonimmediate release formulations including but not limited to sustained release and delayed release formulations.

The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period.

The term “delayed release” is used in its conventional sense to refer to a drug formulation in which there is a time delay between oral administration of the formulation and the release of the drug therefrom. “Delayed release” may or may not involve gradual release of drug over an extended period of time, and thus may or may not be “sustained release.” The “delayed release” formulations herein are enterically coated compositions. “Enteric coating” or “enterically coated” as used herein relates to the presence of polymeric materials in a drug formulation that results in an increase in the dosage form's resistance to degradation in the upper gastrointestinal tract, and/or a decrease in the release or exposure of the drug in the upper gastrointestinal tract.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” sickle cell disease, as the term “treating” is used herein, encompasses treatment of sickle cell disease, or the symptoms associated therewith, e.g., pain, in a clinically symptomatic individual, or the prevention of pain in an asymptomatic individual.

The terms “absorption” and “transmembrane absorption” as used herein refer to the rate and extent to which a substance passes through a body membrane.

### II. Method of the Invention

The invention includes, in one aspect, a method of preventing vascular occlusion, recurrent pain, and/or organ damage associated with sickle cell disease. The method includes administering an effective amount of an active agent to the patient to inhibit the adhesion of sickle erythrocytes to vascular endothelium. Preferably, the active agent is heparin. It has been discovered that there is P-selectin mediated adhesion between, and among, red blood cells, the vascular endothelium, and other circulating blood cells. There is evidence that blocking P-selectin will provide an effective treatment or prevention of pain in sickle cell patients. Considered below are the steps in practicing the invention.

A. Administration of an Active Agent

The method employs an active agent useful for administration to the patient. The active agent inhibits binding of sickle erythrocytes to P-selectin on the patient's vascular endothelium. Several active agents are capable of achieving this inhibition and are contemplated for use in the invention.

i. Basis of Application

While not wishing to be bound by any specific molecular mechanisms underlying the properties of the successful treatment of sickle cell patients, the results of the experiments, as given in Example 1 below, demonstrate that inhibition of P-selectin plays a role. P-selectin is a sticky molecule that promotes the binding of cells to each other. It is a member of the three-member adhesion class called the Selectin Family, each of which functions as a cell adhesion receptor. The family includes also E-selectin and L-selectin. P-selectin was originally discovered on blood cells called platelets, which explains its name. It is also found on the surface of the cells that line the blood vessels of the body (endothelial cells). The appearance of P-selectin on the surface of platelets or endothelial cells occurs only when the cells are activated by a specific stimulant. On the surface of the cell, the molecule mediates specific binding of other cells via molecules called ligands. The best known ligand molecule for P-selectin is P-selectin Glycoprotein Ligand-1 (PSGL-1). E-selectin and L-selectin are found, respectively, on endothelial cells and on white blood cells (leukocytes) which fight infections and mediate inflammation. These molecules too have specific ligands. Selectin ligands share certain of the smaller molecules responsible for their specificity in ligand-selectin interactions and are therefore sometimes blocked by the same blocking agents.

Work conducted by the inventors in support of the present invention has shown that P-selectin is a key component of the abnormal cell adhesion in sickle cell disease. It is an object of the invention to provide agents that block P-selectin binding of sickle cells to provide clinical benefit, particularly treatment of pain, to patients with sickle cell syndromes.

A list of active agents, including heparin, which interfere with P-selectin adhesion and which may be useful in practicing the invention is described below.

ii. Suitable Active Agents

a. Heparin

As noted above, a preferable active agent is heparin. The heparin used in the method of the invention can be either a commercial heparin preparation of pharmaceutical quality or a crude heparin preparation, such as is obtained upon extracting active heparin from mammalian tissues or organs. The commercial product (USP heparin) is available from several sources (e.g., SIGMA Chemical Co., St. Louis, Mo.), generally as an alkali metal or alkaline earth salt (most commonly as sodium heparin). Alternatively, the heparin can be extracted from mammalian tissues or organs, particularly from intestinal mucosa or lung from, for example, beef, porcine and sheep, using a variety of methods known to those skilled in the art (see, e.g., Coyne, Erwin, Chemistry and Biology of Heparin, (Lundblad, R. L., et al. (Eds.), pp. 9-17, Elsevier/North-Holland, N.Y. (1981)). In a preferred embodiment, the heparin is porcine heparin.

Heparin and heparin-like compounds have also been found in plant tissue where the heparin or heparin-like compound is bound to the plant proteins in the form of a complex. Heparin and heparin-like compound derived from plant tissue are of particular importance because they are considerably less expensive than heparin and heparin-like compounds harvested from animal tissue.

Plants which contain heparin or heparin-like compounds such as physiologically acceptable salts of heparin, or functional analogs thereof may also be a suitable source for the present invention. Typical plant sources of heparin or heparin-like compounds include artemisia princeps, nothogenia fastigia (red seaweed), copallina pililifera (red algae), cladophora sacrlis (green seaweed), chaetomorpha anteninna (green seaweed), aopallina officinalis (red seaweed), monostrom nitidum, laminaria japonica, filipendula ulmaria (meadowsweet), ecklonia kuroma (brown seaweed), ascophyllum nodosum (brown seaweed), ginkgo biloba, ulva rigida (green algae), stichopus japonicus (seacucumber), panax ginseng, spiralina maxima, spirulina platensis, laurencia gemmifera (red seaweed), larix (larchwood), and analogs thereof.

The heparin may be low molecular weight heparin (LMWH) or, alternatively, standard or unfractionated heparin. LMWH, as used herein, includes reference to a heparin preparation having an average molecular weight of about 3,000 Daltons to about 8,000 Daltons, preferably about 4,000 Daltons to about 6,000 Daltons. Such LMWHs are commercially available from a number of different sources (e.g., SIGMA Chemical Co., St. Louis, Mo.). The heparin compounds of the present invention can be prepared using a number of different separation or fractionation techniques known to and used by those of skill in the art. Such techniques include, for example, gel permeation chromatography (GPC), high-performance liquid chromatography (HPLC), ultrafiltration, size exclusion chromatography, etc.

LMWHs are currently produced in several different ways: (i) enrichment of LMWH present in standard heparin by fractionation; ethanol and or molecular sieving e.g., gel filtration or membrane filtration; (ii) controlled chemical depolymerization (by nitrous acid, β-elimination or periodate oxidation); and (iii) enzymatic depolymerization by heparinases. The conditions for depolymerization can be carefully controlled to yield products of desired molecular weights. Nitrous acid depolymerization is commonly used. Also employed is depolymerization of the benzylic ester of heparin by β-elimination, which yields the same type of fragment as enzymatic depolymerization using heparinases. Preferably, the heparin is produced by treating porcine heparin with a mixture of heparinases, under conditions effective to produce an average molecular weight of heparin between 4000-6000 Daltons.

LMWHs with low anticoagulant activity and retaining basic structure can be prepared by depolymerization using periodate oxidation. Several LMWHs are available commercially: (i) Fragmin with molecular weight of 4000-6000 Daltons is produced by controlled nitrous acid depolymerization of sodium heparin from porcine intestinal mucosa by Kabi Pharmacia Sweden (see also U.S. Pat. No. 5,686,431 to Cohen et al.); (ii) Fraxiparin and Fraxiparine with an average molecular weight of 4,500 Daltons are produced by fractionation or controlled nitrous acid depolymerzation, respectively, of calcium heparin from porcine intestinal mucosa by Sanofi (Chaoy laboratories); (iii) Lovenox (Enoxaparin and Enoxaparine) is produced by depolymerization of sodium heparin from porcine intestinal mucosa using β-elimination by Farmuka SF France and distributed by Aventis under the trade names Clexane and Lovenox; and (iv) Logiparin (LHN-1, Novo, Denmark) with a molecular weight of 600 to 20,000 Daltons and with more than 70% between 1500 and 10,000 Daltons is produced by enzymatic depolymerization of heparin from intestinal mucosa, using heparinase. See also U.S. Pat. No. 5,534,619 to Wakefield et al. Exemplary low molecular weight heparin fragments include, but are not limited to, enoxaparin, dalteparin, danaproid, gammaparin, nadroparin, ardeparin, tinzaparin, certoparin and reviparin.

In another embodiment, the heparin compounds of the present invention can be obtained from unfractionated heparin by first depolymerizing the unfractionated heparin to yield low molecular weight heparin and then isolating or separating out the fraction of interest. Unfractionated heparin is a mixture of polysaccharide chains composed of repeating disaccharides made up of a uronic acid residue (D-glucuronic acid or L-iduronic acid) and a D-glucosamine acid residue. Many of these disaccharides are sulfated on the uronic acid residues and/or the glucosamine residue. Generally, unfractionated heparin has an average molecular weight ranging from about 6,000 Daltons to 40,000 Daltons, depending on the source of the heparin and the methods used to isolate it.

In a preferred embodiment, the heparin retains an ability to bind P-selectin, but is a non-anticoagulant form. Particularly preferred heparin according to this embodiment include heparin formed by desulfating heparin at the 2-O position of uronic acid residues and/or the 3-O position of glucosamine residues of heparin. Heparin and heparan sulfate consist of repeating disaccharide units containing D-glucuronic acid (GIcA) or L-iduronic acid (IdoA) and a glucosamine residue that is either N-sulfated (GIcNS), N-acetylated (GIcNAc), or, occasionally, unsubstituted (GIcNH2) (Esko, J. D., and Lindahl, U. 2001. Molecular diversity of heparan sulfate. J. Clin. Invest. 108:169-173). The disaccharides may be further sulfated at C6 or C3 of the glucosamine residues and C2 of the uronic acid residues. The potent anticoagulant activity of heparin may depend on a specific arrangement of sulfated sugar units and uronic acid epimers, which form a binding site for antithrombin. See, e.g., Wang, L. et al. (2002) J Clin Invest, July 2002, Volume 110, Number 1, 127-136. 2-O,3-O-desulfated heparin (2/3DS-heparin) may be prepared according to any standard method known in the art, e.g. the method of Fryer, A. et al. (1997) Selective O-desulfation produces nonanticoagulant heparin that retains pharmological activity in the lung. J. Pharmacol. Exp. Ther. 282:208-219. The anticoagulant activity of heparin and modified heparinoids may be analyzed, e.g., by amidolytic anti-factor Xa assay as described in Buchanan, M. R., Boneu, B., Ofosu, F., and Hirsh, J. (1985) The relative importance of thrombin inhibition and factor Xa inhibition to the antithrombotic effects of heparin. Blood 65:198-201.

In another embodiment of the invention, the active agent is a rationally designed LMWH that possess high anti-Xa activity and enriched anti-IIa activity, two to three times that of heparin on a mass basis (Sundaram, M. et al. (2003) Rational design of low-molecular weight heparins with improved in vivo activity Proc. Natl. Acad. Sci. USA, Vol. 100, Issue 2, 651-656). As a result of the enriched anti-Xa and IIa activity for rdLMWH-1 and -2, these molecules may be more effective than are conventional LMWHs. In addition, because of their enriched activity and lower polydispersity, rationally designed LMWHs do not suffer from reduced susceptibility to protamine neutralization. An exemplary method for preparing rationally designed LMWHs is given in Example 3.

b. Additional Active Agents

The active agent of this invention can inhibit interaction between P-selectin and a ligand of P-selectin. By inhibiting interaction is meant, e.g., that P-selectin and its ligand are unable to properly bind to each other to effect proper formation of vascular occlusion. Such inhibition can be the result of any one of a variety of events, including, e.g., preventing or reducing interaction between P-selectin and the ligand, inactivating P-selectin and/or the ligand, e.g., by cleavage or other modification, altering the affinity of P-selectin and the ligand for each other, diluting out P-selectin and/or the ligand, preventing surface, plasma membrane, expression of P-selectin or reducing synthesis of P-selectin and/or the ligand, synthesizing an abnormal P-selectin and/or ligand, synthesizing an alternatively spliced P-selectin and/or ligand, preventing or reducing proper conformational folding of P-selectin and/or the ligand, modulating the binding properties of P-selectin and/or the ligand, interfering with signals that are required to activate or deactivate P-selectin and/or the ligand, activating or deactivating P-selectin and/or the ligand at the wrong time, or interfering with other receptors, ligands or other molecules which are required for the normal synthesis or functioning of P-selectin and/or its ligand.

Examples of active agents include soluble forms of P-selectin or the ligand, inhibitory proteins, inhibitory peptides, inhibitory carbohydrates, inhibitory glycoproteins, inhibitory glycopeptides, inhibitory sulfatides, synthetic analogs of P-selectin or the ligand, certain substances derived from natural products, inhibitors of granular release, and inhibitors of a molecule required for the synthesis or functioning of P-selectin or the ligand.

The soluble form of either P-selectin or the ligand, or a portion thereof, can compete with its cognate molecule for the binding site on the complementary molecule, and thereby reduce or eliminate binding between the membrane-bound P-selectin and the cellular ligand. The soluble form can be obtained, e.g., from purification or secretion of naturally occurring P-selectin or ligand, from recombinant P-selectin or ligand, or from synthesized P-selectin or ligand. Soluble forms of P-selectin or ligand are also meant to include, e.g., truncated soluble secreted forms, proteolytic fragments, other fragments, and chimeric constructs between at least a portion of P-selectin or ligand and other molecules. Soluble forms of P-selectin are described in Mulligan et al., J. Immunol., 151: 6410-6417, 1993, and soluble forms of P-selectin ligand are described in Sako etal., Cell 75(6): 1179-1186, 1993.

Inhibitory proteins include, e.g., anti-P-selectin antibodies (Palabrica et al., Nature 359: 848-851, 1992; Mulligan et al., J. Clin. Invest. 90: 1600-1607, 1992; Weyrich et al., J. Clin. Invest. 91: 2620-2629, 1993; Winn et al., J. Clin. Invest. 92: 2042-2047, 1993); anti-P-selectin ligand antibodies (Sako et al., Cell 75(6): 1179-1186, 1993); Fab (2) fragments of the inhibitory antibody generated through enzymatic cleavage (Palabrica et al., Nature 359: 848-851, 1992); P-selectin-IgG chimeras (Mulligan etal., Immunol., 151: 6410-6417, 1993); and carrier proteins expressing a carbohydrate moiety recognized by P-selectin. The antibodies can be directed against P-selectin or the ligand, or a subunit or fragment thereof. Both polyclonal and monoclonal antibodies can be used in this invention. Preferably, monoclonal antibodies are used. Most preferably, the antibodies have a constant region derived from a human antibody and a variable region derived from an inhibitory mouse monoclonal antibody. Antibodies to human P-selectin are described in Palabrica et al., Nature 359: 848-851,1992; Stone and Wagner, J. C. I., 92: 804-813, 1993; and to mouse P-selectin are described in Mayadas et al., Cell, 74: 541-554, 1993. Antibodies to human ligand are described in Sako et al., Cell 75(6): 1179-1186, 1993. Antibodies that are commercially available against human P-selectin include clone AC1.2 monoclonal from Becton Dickinson, San Jose, Calif.

An inhibitory peptide can, e.g., bind to a binding site on the P-selectin ligand so that interaction as by binding of P-selectin to the ligand is reduced or eliminated. The inhibitory peptide can be, e.g., the same, or a portion of, the primary binding site of P-selectin, (Geng et al., J. Biol. Chem., 266: 22313-22318, 1991, or it can be from a different binding site. Inhibitory peptides include, e.g., peptides or fragments thereof which normally bind to P-selectin ligand, synthetic peptides and recombinant peptides. In another embodiment, an inhibitory peptide can bind to a molecule other than P-selectin or its ligand, and thereby interfere with the binding of P-selectin to its ligand because the molecule is either directly or indirectly involved in effecting the synthesis and/or functioning of P-selectin and/or its ligand.

Inhibitory carbohydrates include oligosaccharides containing sialyl-Lewis a or sialyl-Lewis x or related structures or analogs, carbohydrates containing 2,6 sialic acid, heparin fractions depleted of anti-coagulant activity, heparin oligosaccharides, e.g., heparin tetrasaccharides or low weight heparin, and other sulfated polysaccharides. Inhibitory carbohydrates are described in Nelson et al., Blood 82: 3253-3258, 1993; Mulligan et al., Nature 364: 149-151, 1993; Ball et al., J. Am. Chem. Soc. 114: 5449-5451, 1992; De Frees et al., J. Am. Chem. Soc. 115: 7549-7550, 1993. Inhibitory carbohydrates that are commercially available include, e. g., 3′-sialyl-Lewis x, 3′-sialy-Lewis a, lacto-N-fucopentose III and 3′-sialyl-3-fucosyllactose, from Oxford GlycoSystems, Rosedale, N.Y.

Inhibitory glycoproteins, e.g., PSGL-1, 160 kD monospecific P-selectin ligand, lysosomal membrane glycoproteins, glycoprotein containing sialyl-Lewis x, and inhibitory sulfatides (Suzuki et al., Biochem. Biophys. Res. Commun. 190: 426-434, 1993; Todderud et al., J. Leuk. Biol. 52: 85-88, 1992) that inhibit P-selectin interaction with its ligand can also be used in this invention.

Synthetic analogs or mimetics of P-selectin or the ligand also can serve as agents. P-selectin analogs or mimetics are substances which resemble in shape and/or charge distribution P-selectin. An analog of at least a portion of P-selectin can compete with its cognate membrane-bound P-selectin for the binding site on the ligand, and thereby reduce or eliminate binding between the membrane-bound P-selectin and the ligand. Ligand analogs or mimetics include substances which resemble in shape and/or charge distribution the carbohydrate ligand for P-selectin. An analog of at least a portion of the ligand can compete with its cognate cellular ligand for the binding site on the P-selectin, and thereby reduce or eliminate binding between P-selectin and the cellular ligand. In certain embodiments which use a ligand analog, the sialic acid of a carbohydrate ligand is replaced with a group that increases the stability of the compound yet still retains or increases its affinity for P-selectin, e.g. a carboxyl group with an appropriate spacer. An advantage of increasing the stability is that it allows the agent to be administered orally. Sialyl-Lewis x analog with glucal in the reducing end and a bivalent sialyl-Lewis x anchored on a galactose residue via β-1,3- and β-1,6-linkages also inhibit P-selectin binding (DeFrees et al., J. Am. Chem. Soc., 115: 7549-7550, 1993).

Active agents are also meant to include substances derived from natural products, such as snake venoms and plant extracts, that inhibit P-selectin interaction with its ligand. Such substances can inhibit this interaction directly or indirectly, e.g., through specific proteolytic cleavage or other modification of P-selectin or its ligand.

An inhibitor of granular release also interferes with P-selectin expression on the cell surface, and therefore interferes with P-selectin function. By granular release is meant the secretion by exocytosis of storage granules containing P-selectin: Weibel-Palade bodies of endothelial cells or [agr]-granules of platelets. The fusion of the granular membrane with the plasma membrane results in expression of P-selectin on the cell surface. Examples of such agents include colchicine. (Sinha and Wagner, Europ. J. Cell. Biol. 43: 377-383, 1987).

Active agents also include inhibitors of a molecule that is required for synthesis, post-translational modification, or functioning of P-selectin and/or the ligand, or activators of a molecule that inhibits the synthesis or functioning of P-selectin and/or the ligand. Agents include cytokines, growth factors, hormones, signaling components, kinases, phosphatases, homeobox proteins, transcription factors, translation factors and post-translation factors or enzymes. Agents are also meant to include ionizing radiation, non-ionizing radiation, ultrasound and toxic agents which can, e.g., at least partially inactivate or destroy P-selectin and/or the ligand.

As noted above, in certain embodiments of the invention, the active agent may be monoclonal and/or polyclonal antibodies directed against P-selectin or its ligand PSGL-1. Mouse, or other nonhuman antibodies reactive with P-selectin or its ligand can be obtained using a variety of immunization strategies, such as those described in U.S. Pat. Nos. 6,210,670; 6,177,547; and 5,622,701; each of which is incorporated by reference herein. In some strategies, nonhuman animals (usually nonhuman mammals), such as mice, are immunized with P-selectin antigens. Preferred immunogens are cells stably transfected with P-selectin and expressing these molecules on their cell surface. Other preferred immunogens include P-selectin proteins or epitopic fragments of P-selectin containing the segments of these molecules that bind to the exemplified reacting antibodies.

Antibody-producing cells obtained from the immunized animals are immortalized and selected for the production of an antibody which specifically binds to multiple selectins. See generally, Harlow & Lane, Antibodies, A Laboratory Manual (C.S.H.P. N.Y., 1988) (incorporated by reference for all purposes).

The invention provides humanized antibodies having similar binding specificity and affinity to selected mouse or other nonhuman antibodies. Humanized antibodies are formed by linking CDR regions (preferably CDR1, CDR2 and CDR3) of non-human antibodies to human framework and constant regions by recombinant DNA techniques. See Queen et al., Proc. Natl. Acad. Sci. USA 86:10029-10033 (1989) and WO 90/07861 (incorporated by reference in their entirety). The humanized immunoglobulins have variable region framework residues substantially from a human immunoglobulin (termed an acceptor immunoglobulin) and complementarity determining regions substantially from a mouse immunoglobulin described above (referred to as the donor immunoglobulin). The constant region(s), if present, are also substantially from a human immunoglobulin.

In another embodiment of the invention, human antibodies reactive with P-selectin are provided. These antibodies are produced by a variety of techniques described in the literature, including trioma methodology, transgenic non-human mammals, and phage display methods.

Having produced an antibody having desirable properties, other non antibody agents having similar binding specificity/and or affinity can be produced by a variety of methods. For example, Fodor et al., U.S. Pat. No. 5,143,854, discuss a technique termed VLSIPS, in which a diverse collection of short peptides are formed at selected positions on a solid substrate. Such peptides could then be screened for binding to an epitopic fragment recognized by the antibody. Libraries of short peptides can also be produced using phage-display technology, see, e.g., Devlin W091/18980. The libraries can be screened for binding to an epitopic fragment recognized by the antibody.

Preferred active agents contemplated for use in the invention include heparinoids that block P-selectin binding; the carbohydrate molecule fucoidin and synthetic sugar derivatives such as OJ-R9188 which block selectin-ligand interactions; the carbon-fucosylated derivative of glycyrrhetinic acid GM2296 and other sialyl Lewis X glycomimetic compounds; inhibitors of P-selectin expression such as mycophenolate mofetil, the proteasome inhibitor ALLN, and antioxidants such as PDTC; sulfatide and sulfatide analogues such as BMS-190394; the 19 amino acid terminal peptide of PSGL1, other PSGL-1 peptides, PSGL-1 fusion proteins, PSGL-1 analogues, and selective inhibitors of PSGL-1 binding such as beta-C-mannosides; benzothiazole compounds derived from ZZZ21322 such as Compound 2; and/or statins, particularly Simvastatin which is marketed by Merck as Zocor.

iii. Enhancers

In certain embodiments, the invention contemplates the use of enhancers, e.g. liposomes and/or nanocapsules for the delivery of an active agent or active agents, such that the active agent is complexed with an enhancer compound effective to enhance the uptake of the heparin from the gastrointestinal (GI) tract into the bloodstream. Such formulations may be preferred for the introduction of pharmaceutically-acceptable formulations of the heparins, antibodies, and/or other active agents disclosed herein. The formation and use of liposomes is generally known to those of skill in the art. See, e.g., Backer, M. V., et al. (2002) Bioconjug Chem 13(3):462-7.

In one embodiment, 1-(acyloxyalkyl)imidazoles (AAI) are of use in the instant invention as nontoxic, pH-sensitive liposomes. AAI are incorporated into the liposomes as described in Chen, F, et al. (2003) Cytosolic delivery of macromolecules: I. Synthesis and characterization of pH-sensitive acyloxyalkylimidazoles Biochimica et Biophysica Acta (BBA)—Biomembranes Volume 1611, Issues 1-2, pp 140-150. Exemplary 1-(acyloxyalkyl)imidazoles (AAI) may be synthesized by nucleophilic substitution of chloroalkyl esters of fatty acids with imidazole. The former may be prepared from fatty acid chloride and an aldehyde. When incorporated into liposomes, these lipids show an apparent pKa value ranging from 5.12 for 1-(palmitoyloxymethyl)imidazole (PMI) to 5.29 for 1-[(α-myristoyloxy)ethyl]imidazole (α-MEI) as determined by a fluorescence assay. When the imidazole moiety is protonated, the lipids are surface-active, as demonstrated by hemolytic activity towards red blood cells. AAI may be hydrolyzed in serum as well as in cell homogenate. They are significantly less toxic than biochemically stable N-dodecylimidazole (NDI) towards Chinese hamster ovary (CHO) and RAW 264.7 (RAW) cells as determined by MTT assay.

A number of absorption enhancers are known in the art and may be utilized in the invention. For instance, medium chain glycerides have demonstrated the ability to enhance the absorption of hydrophilic drugs across the intestinal mucosa (Pharm. Res. Vol 11:1148-54 (1994)). Sodium caprate has been reported to enhance intestinal and colonic drug absorption by the paracellular route (Pharm. Res. 10:857-864 (1993); Pharm. Res. 5:341-346 (1988)). U.S. Pat. No. 4,545,161 discloses a process for increasing the enteral absorbability of heparin and heparinoids by adding non-ionic surfactants such as those that can be prepared by reacting ethylene oxide with a fatty acid, a fatty alcohol, an alkylphenol or a sorbitan or glycerol fatty acid ester.

U.S. Pat. No. 3,510,561 to Koh et al. describes a method for enhancing heparin absorption through mucous membranes by co-administering a sulfone and a fatty alcohol along with the heparin. U.S. Pat. No. 4,239,754 to Sache et al. describes liposomal formulations for the oral administration of heparin, intended to provide for a prolonged duration of action. The heparin is retained within or on liposomes, which are preferably formed from phospholipids containing acyl chains deriving from unsaturated fatty acids.

U.S. Pat. No. 4,654,327 to Teng pertains to the oral administration of heparin in the form of a complex with a quaternary ammonium ion. U.S. Pat. No. 4,656,161 to Herr describes a method for increasing the enteral absorbability of heparin or heparinoids by orally administering the drug along with a non-ionic surfactant such as polyoxyethylene-20 cetyl ether, polyoxyethylene-20 stearate, other polyoxyethylene (polyethylene glycol)-based surfactants, polyoxypropylene-1 5 stearyl ether, sucrose palmitate stearate, or octyl-β-D-glucopyranoside. U.S. Pat. No. 4,695,450 to Bauer describes an anhydrous emulsion of a hydrophilic liquid containing polyethylene glycol, a dihydric alcohol such as propylene glycol, or a trihydric alcohol such as glycerol, and a hydrophobic liquid, particularly an animal oil, a mineral oil, or a synthetic oil.

U.S. Pat. No. 4,703,042 to Bodor describes oral administration of a salt of polyanionic heparinic acid and a polycationic species. U.S. Pat. No. 4,994,439 to Longenecker et al. describes a method for improving the transmembrane absorbability of macromolecular drugs such as peptides and proteins, by co-administering the drug along with a combination of a bile salt or fusidate or derivative thereof and a non-ionic detergent (surfactant). U.S. Pat. No. 5,688,761 to Owen et al. focuses primarily on the delivery of peptide drugs using a water-in-oil microemulsion formulation that readily converts to an oil-in-water emulsion by the addition of an aqueous fluid, whereby the peptide or other water-soluble drug is released for absorption by the body. U.S. Pat. Nos. 5,444,041, 5,646,109 and 5,633,226 to Owen et al. are also directed to water-in-oil microemulsions for delivering biologically active agents such as proteins or peptides, wherein the active agent is initially stored in the internal water phase of the emulsion, but is released when the composition converts to an oil-in-water emulsion upon mixing with bodily fluids.

U.S. Pat. No. 5,714,477 to Einarsson describes a method for improving the bioavailability of heparin, heparin fragments or their derivatives by administering the active agent in combination with one or several glycerol esters of fatty acids. U.S. Pat. No. 5,853,749 to New describes a formulation for buffering the gut to a pH in the range of 7.5 to 9 by coadministering a biologically active agent with a bile acid or salt and a buffering agent. Muranishi (1990), “Absorption Enhancers,” Critical Reviews in Therapeutic Drug Carrier Systems 7 (1):1-33, provides an overview of absorption enhancing compounds for macromolecular drugs. Among the numerous enhancing compounds mentioned are medium chain fatty acids (C(6)-C(12)) such as sodium caprate, and medium chain monoglycerides such as glyceryl-1-monocaprate, dicaprate and tricaprate. Aungst (2000), “Intestinal Permeation Enhancers,” J Pharm. Sci. 89(4):429-442, provides an overview of compounds and methods for enhancing intestinal permeation of drugs, and mentions, for example, fatty acids, surfactants and medium-chain glycerides.

Preferred enhancers include sodium N-[8-(2-hydroxybenzoyl)amino] caprylate (SNAC) and sodium N-[8-(2-hydroxybenzoyl)amino] decanoate (SNAD), as described in U.S. Pat. Nos. 6,525,020, 6,461,643; 6,440,929; 6,344,213; and 5,650,386 each of which is incorporated by reference herein. These enhancers have the advantage of being capable of delivering active agents of the invention through various chemical, physical, and biological barriers such as the GI tract and are also well suited for delivering active agents which are subject to environmental degradation.

Polymerized liposomes are oral drug delivery systems used to deliver drugs to the mucosal tissue of the intestine and other epithelial surfaces which utilizes polymerized liposomes as the active agent carriers. The polymerizable fatty acids and phospholipids are used to prepare liposomes with significant stability in the GI tract. The polymerizable fatty acids are used to improve the preparation and loading of the polymerized liposomes. The polymerized liposomes prepared using these novel fatty acids or phospholipids are especially useful as active agent carriers. U.S. Pat. No. 6,187,335, which is incorporated herein by reference, describes the fatty acids and phospholipids, how they are prepared and how they can be utilized to prepare stable polymerized liposomes.

Promdas and Locdas from Elan are enhancers that may be used in the instant invention. U.S. Pat. No. 6,423,334, which is incorporated herein by references, provides a composition having a non-ionic vegetable oil GI tract absorption enhancer for increasing the enteral absorbability of drugs, especially oral absorbability of hydrophilic and macromolecular drugs. The non-ionic vegetable oil GI tract absorption enhancer is capable of enhancing the uptake of a drug from the gastrointestinal tract so as to allow therapeutically effective amounts of the drug to be transported across the GI tract of an animal such as a human without significant toxic side effects.

U.S. Pat. Nos. 6,468,559; 6,458,383; 6,451,339; 6,383,471; 6,309,663; 6,294,192; 6,267985; and 6,258,363, to Lipocine, each of which is incorporated herein by reference, describe various oral enhancers that may be used in the invention. Preferred commerically available oral enhancers for use in the invention include Hydroance and/or Lipral from Lipocine (http://www.lipocine.com/thydroance.htm).

U.S. Pat. Nos. 6,495,530 and 6,255,296, each of which is incorporated herein by reference, describes various formulations that may be of use in the present invention.

Additional commercially available enhancers which may find use in the instant invention include Labrasol (caprylocaproyl macrogolglycerides), TPGS (D-α-tocopheryl polyethylene glycol 1000 succinate), DOCA, which enhances hydrophobicity of conjugated agent, alginate/poly-L-lysine microparticles, polycarbophil, hydroxypropyl methylcellulose, carbopol 934, sodium salicylate, polyoxyethylene-9-lauryl ether, poly(ethylcyanoacrylate), 2-alkoxy-3-alkylamidopropylphosphocholines, 2-alkoxy-3-alkylamidopropylphosphocholines, poly(diethyl)methylidenemalonate and/or dodecylphosphocholine.

In one embodiment, the present dosage forms are delayed release in nature, such that the release of composition from the dosage form is delayed after oral administration, and preferably occurs in the lower GI tract. After reaching the intended release site, there may or may not be a further mechanism controlling release of the composition from the dosage form. That is, delayed release of the composition from the dosage form may be immediate and substantially complete at the intended release site, or, alternatively, release at the intended site may occur in a sustained fashion over an extended period of time, or in a staged or pulsatile fashion.

Nanocapsules can generally entrap compounds in a stable and reproducible way (Whelan, J. (2001) Drug Discov Today 6(23):1183-84). To avoid side effects due to intracellular polymeric overloading, such ultrafine particles (sized around 0.1 μm) may be designed using polymers able to be degraded in vivo. Biodegradable polyisobutylcyanoacrylate nanoparticles that meet these requirements are contemplated for use in the present invention, and such particles may be easily made, as described in, e.g. Lambert, G., et al. (2001) Int J Pharm 214(1-2):13-6. Methods of preparing polyalkyl-cyano-acrylate nanoparticles containing biologically active substances and their use are described in U.S. Pat. Nos. 4,329,332, 4,489,055 and 4,913,908 which are incorporated by reference herein.

Pharmaceutical compositions containing nanocapsules for the delivery of active agents are described in U.S. Pat. Nos. 5,500,224 and 5,620,708. U.S. Pat. No. 5,500,224 describes a pharmaceutical composition in the form of a colloidal suspension of nanocapsules comprising an oily phase consisting essentially of an oil containing dissolved therein a surfactant and suspended therein a plurality of nanocapsules having a diameter of less than 500 nanometers. U.S. Pat. No. 5,620,708 describes compositions and methods for the administration of drugs and other active agents. The compositions comprise an active agent carrier particle attached to a binding moiety which binds specifically to a target molecule present on the surface of a mammalian enterocyte. The binding moiety binds to the target molecule with a binding affinity or avidity sufficient to initiate endocytosis or phagocytosis of the particulate active agent carrier so that the carrier will be absorbed by the enterocyte. The active agent will then be released from the carrier to the host's systemic circulation. In this way, degradation of degradation-sensitive drugs, such as polypeptides, in the intestines can be avoided while absorption of proteins and polypeptides from the intestinal tract is increased. Alternatively, the invention contemplates release of the active agent in the environment surrounding the blood cell. For example, in one embodiment, heparin is released from the nanocapsule following target moiety binding to the target cell, such that heparin is released into the microenvironment surrounding the target cell, e.g. a red blood cell. U.S. Pat. Nos. 6,379,683 and 6,303,150 describe methods of making nanocapsules and the use thereof, and are incorporated herein by reference.

Additional delivery agents such as small unilamellar vesicles (suv's), as described in U.S. Pat. No. 6,180,114, which is incorporated herein by reference in its entirety, may be employed in the present invention.

iv. Inhibition of the Binding of P-Selectin

As described above, the active agent is administered in an amount effective to inhibit binding of sickle erythrocytes to P-selectin, e.g. the P-selectin on the vascular endothelium. This binding inhibition may be assayed by a number of methods known in the art. See, e.g., Frangos, J. A., et al. (1988) Shear stress induced stimulation of mammalian cell metabolism, Biotechnology and Bioengineering 32:1053-1060.

In one exemplary embodiment, as shown in Example 2 below, the inhibition in binding is evidenced by a reduction or prevention of binding of of sickle red blood cells to cultured human endothelial cell (HUVEC) monolayers in vitro.

A variety of in vivo animal models can also be used to evaluate the ability of the active agents of the invention to treat sickle cell disease or the symptoms associated therewith (in addition to the in vitro test described above). See, e.g., Martinez-Ruiz, R, et al. (2001) Inhaled nitric oxide improves survival rates during hypoxia in a sickle cell (SAD) mouse model, Anesthesiology Jun;94(6):1113-8 and Embury S. H., et al. (1999) In vivo blood flow abnormalities in the transgenic knockout sickle cell mouse, J Clin Invest. Mar;103(6):915-20. In a preferred embodiment, the inhibition of binding is evidenced by enhance microvascular blood flow in the mucosal-intestinal blood vessels of transgenic knockout sickle cell mice in vivo. This enhancement may include, e.g., restoration of blood flow velocity that has been slowed by topical application of thrombin receptor agonist peptide-1 (TRAP-1) by use of topical application of heparin onto the mesentery; and/or prevention of slowing of microvascular blood flow by topical application of TRAP-1 by use of pretreatment of the mouse with intravenous heparin.

A number of in vivo patient evaluation methods for monitoring or measuring binding inhibition may also be used. In one embodiment of the invention, the enhancement microvascular blood flow in conjunctivae of patients with sickle cell disease is monitored. Such monitoring may include computer-assisted intravital microscopy in vivo. Alternatively, the velocity of microvasculat flow may be monitored using Laser-Doppler velocimetry. See, e.g., Rodgers etal.,NEJM311:1534,1984; and Brody et al., Am J Radiol 151:139,1988, both of which are incorporated herein by reference.

The binding inhibition may also be monitored by the promotion or enhancement of vascular well-being in patients with sickle cell disease. This well-being may be determined by surrogate markers of vascular endothelial well-being, sickle cell (sickle RBC) sickling, monocyte activation, platelet activation, coagulation, and/or fibrinolysis.

Surrogate markers of vascular endothelial well-being include, but are not limited to, soluble P-selectin (sP-sel), vascular endothelial cell adhesion molecule-1 (sVCAM-1), tumor necrosis factor-a (TNFa), Interleukin-1b (IL-1b), IL-6, IL-8, IL-10, a2-macroglobulin, C-reactive protein (CRP), high sensitivity CRP, soluble interleukin-2 receptor (slL-2R), substance P, endothelin-1, circulating endothelial cells (CEC), microparticles (MP) from the plasma membranes of endothelial cells, MP from monocytes, platelets, and sickle RBC. Markers for sickle cell sickling include MP from sickle RBC. Markers for monocyte activation include MP from monocytes. Markers for platelet activation include, β-thromboglobulin (βP-TG), platelet factor-4 (PF-4), and MP from platelets. Markers for coagulation include fibrinopeptide A (FPA), fragment 1.2 (F1.2), and thrombin-antithrombin complexes (TAT). Markers for fibrinolysis include D-dimers and plasmin-antiplasmin complexes (PAP).

In another embodiment of the invention, the binding inhibition is measured by a reduction in frequency or prevention of pain crises during long-term administration to patients with sickle cell disease in vivo.

In yet another embodiment, the inhibition in binding is evidenced by a reduction in the adhesion of sickle erythrocytes in a patient blood sample to human umbilical vein endothelial cells in vitro, relative to patient cell binding prior to treatment.

In certain embodiments of the invention, the inhibition is evidenced by at least a 5% reduction, preferably at least 25%, more preferably at least 50%, even more preferably at least 75%, and yet even more preferably 90 to 100% reduction in sickle erythrocytes to endothelial cells.

By administering the active agents as described above, the adhesion of sickle erythrocytes to vascular endothelium in the patient is inhibited. Thus, patient pain associated with vascular occlusion is decreased and/or preferably, prevented.

### III. Pharmaceutical Compositions

The active agents of this invention can be incorporated into a variety of formulations for therapeutic administration. More particularly, the active agents can be formulated into pharmaceutical compositions by combination with appropriate, pharmaceutically acceptable carriers or diluents, and may be formulated into various preparations, preferably in liquid forms, such as slurries, and solutions. Administration of the active agent is preferably achieved by oral administration.

Suitable formulations for use in the present invention may be found in Remington's Pharmaceutical Sciences (Mack Publishing Company, Philadelphia, Pa., 19th ed. (1995)), the teachings of which are incorporated herein by reference. Moreover, for a brief review of methods for drug delivery, see, Langer, et al (1990) Science 249:1527-1533, the teachings of which are incorporated herein by reference. The pharmaceutical compositions described herein can be manufactured in a manner that is known to those of skill in the art, i.e., by means of conventional mixing, dissolving, levigating, emulsifying, entrapping or lyophilizing processes. The following methods and excipients are merely exemplary and are in no way limiting.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

The pharmaceutical compositions of the present invention may be manufactured using any conventional method, e.g., mixing, dissolving, granulating, levigating, emulsifying, encapsulating, entrapping, melt-spinning, spray-drying, or lyophilizing processes. However, the optimal pharmaceutical formulation will be determined by one of skill in the art depending on the route of administration and the desired dosage. Such formulations may influence the physical state, stability, rate of in vivo release, and rate of in vivo clearance of the administered agent. Depending on the condition being treated, these pharmaceutical compositions may be formulated and administered systemically or locally.

The pharmaceutical compositions of the invention can also be administered by a number of routes, including without limitation, topically, rectally, orally, vaginally, nasally, transdermally. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments.

The pharmaceutical compositions are formulated to contain suitable pharmaceutically acceptable carriers, and may optionally comprise excipients and auxiliaries that facilitate processing of the active compounds into preparations that can be used pharmaceutically. The administration modality will generally determine the nature of the carrier. For tissue or cellular administration, penetrants appropriate to the particular barrier to be permeated are used in the formulation. Such penetrants are generally known in the art. For certain preparations the formulation may include stabilizing materials, such as polyols (e.g., sucrose) and/or surfactants (e.g., nonionic surfactants), and the like.

Preferably, as noted above, the pharmaceutical compositions comprising the agent in dosages suitable for oral administration can be formulated using pharmaceutically acceptable carriers well known in the art. The preparations formulated for oral administration may be in the form of tablets, pills, capsules, cachets, lozenges, liquids, gels, syrups, slurries, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets. Oral formulations may employ liquid carriers such as buffered aqueous solutions, suspensions, and the like.

These preparations may contain one or excipients, which include, without limitation: a) diluents such as sugars, including lactose, dextrose, sucrose, mannitol, or sorbitol; b) binders such as magnesium aluminum silicate, starch from com, wheat, rice, potato, etc.; c) cellulose materials such as methyl cellulose, hydroxypropyhnethyl cellulose, and sodium carboxymethyl cellulose, polyvinyl pyrrolidone, gums such as gum arabic and gum tragacanth, and proteins such as gelatin and collagen; d) disintegrating or solubilizing agents such as cross-linked polyvinyl pyrrolidone, starches, agar, alginic acid or a salt thereof such as sodium alginate, or effervescent compositions; e) lubricants such as silica, talc, stearic acid or its magnesium or calcium salt, and polyethylene glycol; f) flavorants, and sweeteners; g) colorants or pigments, e.g., to identify the product or to characterize the quantity (dosage) of active agent; and h) other ingredients such as preservatives, stabilizers, swelling agents, emulsifying agents, solution promoters, salts for regulating osmotic pressure, and buffers.

The pharmaceutical composition may be provided as a salt of the active agent, which can be formed with many acids, including but not limited to hydrochloric, sulfuric, acetic, lactic, tartaric, malic, succinic, etc. Salts tend to be more soluble in aqueous or other protonic solvents that are the corresponding free base forms.

As noted above, the characteristics of the agent itself and the formulation of the agent can influence the physical state, stability, rate of in vivo release, and rate of in vivo clearance of the administered agent. Such pharmacokinetic and pharmacodynamic information can be collected through pre-clinical in vitro and in vivo studies, later confirmed in humans during the course of clinical trials. Thus, for any compound used in the method of the invention, a therapeutically effective dose in mammals, particularly humans, can be estimated initially from biochemical and/or cell-based assays. Then, dosage can be formulated in animal models to achieve a desirable therapeutic dosage range that modulates P-selectin binding, and/or decreases or prevents pain or other symptoms associated with sickle cell disease.

Toxicity and therapeutic efficacy of such compounds can be determined by standard pharmaceutical procedures in cell cultures such as in vitro human umbilical vein endothelial cells or experimental animals, e.g., for determining the LD50 (the dose lethal to 50% of the population) and the ED50 (the dose therapeutically effective in 50% of the population).

For the method of the invention, any effective administration regimen regulating the timing and sequence of doses may be used. Doses of the active agent preferably include pharmaceutical dosage units comprising an effective amount of the agent.

Typically, the active product, e.g., the heparin compounds, will be present in the pharmaceutical composition at a concentration ranging from about 1 mg per dose to 3,000 mg per dose and, more preferably, at a concentration ranging from about 40 mg (10,000 units) per dose to about 2,700 mg (300,000 units) per dose, more preferably about 50 mg per dose to about 600 mg per dose. In one embodiment, the active agent is administered in a tablet or capsule designed to increase the absorption from the GI tract. In another embodiment, the active agent is contained in a solid or capsule form suitable for oral administration in total dosages between about 50 mg to about 500 mg, and preferably in total dosages of 50 mg (6,250 units), 100 mg (12,500 units), 250 mg (31,250 units) or 500 mg (62,500 units).

Daily dosages may vary widely, depending on the specific activity of the particular active agent. Depending on the route of administration, a suitable dose may be calculated according to body weight, body surface area, or organ size. The final dosage regimen will be determined by the attending physician in view of good medical practice, considering various factors that modify the action of drugs, e.g., the agent's specific activity, the severity of the disease state, the responsiveness of the patient, the age, condition, body weight, sex, and diet of the patient, the severity of any infection, and the like. Additional factors that may be taken into account include time and frequency of administration, drug combination(s), reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in clinical trials. Appropriate dosages may be ascertained through use of established assays for determining concentration of the agent in a body fluid or other sample together with dose response data.

The frequency of dosing will depend on the pharmacokinetic parameters of the agent and the route of administration. Dosage and administration are adjusted to provide sufficient levels of the active agent or to maintain the desired effect. Accordingly, the pharmaceutical compositions can be administered in a single dose, multiple discrete doses, continuous infusion, sustained release depots, or combinations thereof, as required to maintain desired minimum level of the agent.

Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks.

Compositions comprising an active agent of the invention formulated in a pharmaceutical acceptable carrier may be prepared, placed in an appropriate container, and labeled for treatment of an indicated condition. Conditions indicated on the label may include, but are not limited to, treatment of sickle cell disease and prevention of symptoms. Kits are also contemplated, wherein the kit comprises a dosage form of a pharmaceutical composition and a package insert containing instructions for use of the composition in treatment of a medical condition.

Generally, the active agents used in the invention are administered to a subject in an effective amount. Generally, an effective amount is an amount effective to (1) reduce the symptoms of the disease sought to be treated, (2) induce a pharmacological change relevant to treating the disease sought to be treated, and/or (3) prevent the symptoms of the disease sought to be treated.

In addition to being useful in pharmaceutical compositions for the treatment of the sickle cell conditions described above, one of skill in the art will readily appreciate that the active agents, e.g., the heparin compounds, can be used as reagents for elucidating the mechanisms of sickle cell disease in vitro.

From the foregoing, it can be seen how various objects and features of the invention are met.

### IV Examples

The following examples further illustrate the invention described herein and are in no way intended to limit the scope of the invention.

### EXAMPLES 1

P-selectin Mediates the Adhesion of Sickle Erythrocytes to the Endothelium

A. Blood Samples

Heparinized blood samples were obtained from subjects with sickle cell disease and from healthy control subjects with approval of the Committee on Human Research of the University of California, San Francisco.

B. Thrombin Treatment of Endothelial Monolayers, Static Gravity Adherence with Dip Rinse, and Adherence Inhibition Assays.

Thrombin treatment of human umbilical vein endothelial cells (HUVECs) and the static gravity adherence assay with dip rinse were performed as previously described. When 90% confluent, HUVECs (Clonetics, San Diego, Calif.) were treated with 0.1 U/mL thrombin (Sigma Chemicals, St Louis, Mo.) or medium alone for 5 minutes before assaying erythrocyte adherence. Adherent RBCs were counted microscopically in 8 randomly selected 0.15-mm2 fields for each study condition. The adherence data may be presented as percent adherence where 100% is the mean adherence of nonsickle RBCs to untreated HUVECs. The adherent RBCs observed in the microscopic gravity adherence assay are biconcave disks, which is consistent with the observation by several laboratories that the most adhesive sickle cells are the less dense fraction that is relatively devoid of irreversibly sickled cells.

Because of potential modulatory effects of heparin on adherence, the static adherence assay was used to compare the adhesivity of sickle cells and autologous plasma according to whether they were prepared with citrate anticoagulant or with heparin. No significant difference was detected in these 2 anticoagulants. The contribution of P-selectin to thrombin-enhanced adherence was determined by comparing the effects on adherence of exposing HUVECs to no antibody, a 1:200 dilution of nonblocking P-selectin monoclonal antibodies (mAbs) AC1.2 (BD Pharmingen, San Diego, Calif.), or a 1:200 dilution of blocking P-selectin mAb 9E1 (R & D Systems, Minneapolis, Minn.). The contribution of P-selectin to thrombin-enhanced adherence was confirmed by comparing the effects on adherence of adding to each well medium alone, 100 μM sialyl Lewis X (sLeX) tetrasaccharide (sLeX, Sigma), or 500 μM 3′- sialyl-lactose (sLac, Glycotech, Rockville, Md.), an analogous sugar that does not bind P-selectin.

C. Flow Cytometry

Primary antibodies were used for indirect immunofluorescence staining of erythrocytes in flow cytometry the mAbs AC1.2 and 9E1, which are specific for P-selectin. For each mAb an isotype-matched nonspecific mAb or the secondary antibody alone was used as negative controls. The secondary antibody was goat anti-mouse or goat anti-rat IgG conjugated to biotin (Sigma), which was reacted with fluorescein-conjugated Neutravidin (Molecular Probes, Eugene, Oreg.). Fluorescence intensity of 20,000 cells/experiment was quantified on a FACS-scan flow cytometry system and analyzed using Cell-Quest software (Becton Dickinson, San Jose, Calif.).

D. Nonstatic Adherence to Immobilized Sialic Acid-binding Lectin-Ig Chimeras or P-selectin-Ig Chimeras Using a Rotatory Adherence Assay

400 ng bovine serum albumin (BSA; Sigma), a sialic acid-binding lectin (Siglec)-6-Ig chimera, mutated Siglec-7-Ig chimera, or P-selectin-Ig chimera was immobilized on microtiter wells. Siglec-6 is a sialic acid-binding lectin of the immunoglobulin superfamily that does not bind P-selectin ligands or erythrocytes, which was used as negative controls. Whereas Siglec-7 does bind RBCs, the mutated Siglec-7 used as a negative control does not. These chimeras were applied to the wells in 10 mM carbonate buffer overnight and then blocked with 0.5% BSA in Hanks buffered salt solution (University of California, San Francisco, Cell Culture Facility) before incubation with RBCs using a published rotatory adhesion assay. Erythrocyte adherence to BSA, Siglec-6, mutant Siglec-7, or P-selectin was measured as the number of RBCs observed in 8 random 0.04-mm2 fields for each condition. The adherence data are presented as percent adherence where 100% is the mean number of untreated nonsickle erythrocytes/field in a well in which BSA or Siglec was immobilized.

E. Enzyme Treatment of Test RBCs

To determine whether sialic acid on erythrocytes is a recognition determinant for P-selectin, prior to testing RBC adherence the erythrocytes were treated with sialidase using a published method. Packed RBCs were mixed with an equal volume of buffer or 0.1 U/mL Vibrio cholerae sialidase (Calbiochem, San Diego). No hemolysis was detected with 0.1 U/mL sialidase as assayed by colorimetric spectrophotometry. Efficacy of sialidase on RBCs was confirmed by agglutination with peanut extract lectin (Arachis hypogea lectin, Sigma).

F. Statistical Analyses

For each experiment mean adherence was set arbitrarily at 100% for the control data. The mean adherence of data derived from perturbations of control conditions were calculated as a percentage relative to the control. The average of the means from replicate experiments was then calculated. The uncertainty of the estimate of the means from the data distributed in each set is described as SEM. An SEM of 0% resulted when control data sets were normalized to 100%. We used the paired one-tailed Studentt test to compare changes in adhesion resulting from different perturbations of the system.

G. Effect of P-selectin mAb on Erythrocyte Adherence to Endothelial Cells

The effects of the P-selectin blocking mAb 9E1 on the static adherence of nonsickle or sickle RBCs to untreated or thrombin-treated HUVEC monolayers were assessed. The data are consistent with previous reports that the adherence of sickle cells to untreated endothelial cells is greater than that of nonsickle RBCs18,46 and that the adherence of both RBC types to thrombin-treated endothelium is increased compared to untreated endothelium. Blocking endothelial monolayers with mAb 9E1 reduced the adherence of nonsickle RBCs by 21% to untreated endothelium (100%±0% to 79%±11%; P=0.046) and 51% to thrombin-treated endothelium (266%±72% to 131%±34%; P=0.006). Blocking with mAb 9E1 reduced the adherence of sickle cells by 30% to untreated endothelium (132%±0% to 93%±11%; P=0.002) and by 76% to thrombin-treated endothelium (490%±188% to 119%±18%; P=0.038). These reductions in adherence were statistically significant but partial. The persistence of a portion of adhesivity after blocking P-selectin reveals that other adhesion mechanisms also are involved.

The data reveal the static adherence of RBCs to HUVECs that were treated with thrombin or medium alone and then exposed to medium with or without blocking P-selectin antibody 9E1. Further evidence for the involvement of other pathways was derived from a single titration experiment in which we tested the effect of 1:2000, 1:200, and 1:20 dilutions of mAb 9E1 on erythrocyte adherence to thrombin-activated HUVECs. The adherence of nonsickle and sickle RBCs was reduced, respectively, 64% and 70% by a 1:2000 dilution of the mAb, 72% and 84% by a 1:200 dilution, and 66% and 83% by a 1:20 dilution. The persistence of similar levels of adherence at our standard 1:200 mAb dilution and at a 10-fold higher titer of 1:20 further supports the involvement of other adhesion pathways.

To verify the specificity of P-selectin blocking, 3 replicate experiments compared the effects of a pair of isotype-matched P-selectin mAbs, the blocking mAb 9E1 and the nonblocking mAb AC1.2, on RBC adhesion to thrombin-treated endothelial cell monolayers (data not shown). Treatment with AC1.2 resulted in no significant reduction in the adherence of either nonsickle (148%±25% to 160%±6%; P=0.227) or sickle (318%±29% to 359%±25%; P=0.242) RBCs from that observed without mAb. Compared to the adherence observed with AC1.2, treatment of endothelial cells with 9E1 reduced adherence by 48% for nonsickle cells (160%±6% to 84%±23%; P=0.005) and 58% for sickle cells (359%±25% to 150%±38%; P=0.039). Although the exact number of adherent erythrocytes and fractional inhibition varied among different experiments, different patients, patient status, and preparations of HUVECs, we consistently found statistically significant adhesion of both nonsickle and sickle RBCs to endothelial P-selectin.

These data taken together provide evidence for the novel adherence of normal and, to a greater degree, sickle erythrocytes to P-selectin on activated endothelial cells. They also support the contribution of P-selectin-independent pathways in thrombin-enhanced adherence.

H. Effect of sLeX Tetrasaccharide on Erythrocyte Adherence to Endothelial Cells

The sLeX antigen is a recognition determinant for selectins that selectively inhibits their adhesivity; sLac is a saccharide that is structurally related to sLeX but does not bind to P-selectin. Four replicate experiments compared the static adherence of RBCs to endothelial cells in the absence of either saccharide, the presence of sLac, and the presence of sLeX. Adherence to untreated endothelial cells with no added saccharide was not reduced significantly by the addition of sLac for either nonsickle (100%±0% to 105%±15%; P=0.373) or sickle cells (143%±0% to 144%±44%; P=0.493). Neither did sLac reduce significantly the adherence to thrombin-treated endothelial cells for either nonsickle (145%±31% to 162%±28%; P=0.207) or sickle cells (325%±73% to 304%±74%; P=0.240). The inhibitory effect of sLex on adherence was not significant with untreated endothelial cells but significant with thrombin-treated cells. Although not significant, compared with the adherence to untreated endothelial cells with sLac present, the addition of sLeX reduced adherence by an estimated 48% for nonsickle (105%±15% to 55%±12%; P=0.063) and 37% for sickle cells (144%±44% to 91±8%; P=0.121). Compared with the adherence to thrombin-treated endothelial cells when sLac is present, adherence was reduced significantly by the addition of sLeX by 49% for nonsickle (162%±28% to 82%±41%; P=0.047) and 36% for sickle cells (304%±74% 196%±38%; P=0.037). These findings are consistent with the adhesion of nonsickle and sickle erythrocytes to P-selectin on activated endothelial cells. The incomplete inhibition observed with sLeX is not surprising because the inhibitory potency of this saccharide for P-selectin, while specific, is not strong. The absence of an inhibitory effect by sLac confirms the specificity of sLeX for P-selectin in the static adhesion assay.

Although it is reasonable to conclude that the P-selectin mAb 9E1 and sLeX affect the same molecular interaction, we directly tested this precept in our system. We performed an experiment in which we compared the effects of these inhibitors singly and in combination. RBC adherence to thrombin-treated endothelial cells was inhibited nearly identically by mAb 9E1, sLeX, and their combination for nonsickle cells (17%-22%) and for sickle cells (43%-47%). The lack of additive effect is consistent with the knowledge that sLeX and the P-selectin participate in the same molecular interaction. Because neither inhibitor nor their combination completely abrogated adherence, these results too are consistent with the existence of a P-selectin independent pathway for adhesion to thrombin-treated endothelial cells.

In our studies of mAb 9E1 only endothelial cells were exposed to the blocking agent, but in these studies of sLeX both endothelial and RBCs were exposed to the inhibitor. To assess whether the P-selectin blocked by sLeX may also have been on RBCs, we performed a flow cytometry study of nonsickle and sickle RBCs using P-selectin mAbs AC1.2 and 9E1. We detected no signal indicative of the presence of P-selectin on either type of erythrocyte, which is consistent with prior reports of the absence of P-selectin from erythroid cells.

I. Adherence of Erythrocytes to Immobilized Recombinant P-selectin

Multiple molecular mechanisms have been described for sickle cell adherence to unperturbed and activated endothelium. Our results from these studies on thrombin-activated HUVECs also may reflect the participation of adhesion molecules besides P-selectin, such as 1- and 3-integrins dissociated from their matrix binding sites, and unmasked matrix proteins.54 As with leukocyte-endothelial interactions, the actual binding we observe may involve other molecules as well. Yet, selectin interactions typically initiate such adhesion cascades and are critically required. To directly confirm the role of P-selectin in sickle cell adhesion, we tested the nonstatic adhesion of RBCs to a recombinant P-selectin-Ig chimera immobilized on plastic microtiter wells using a rotatory adherence assay. We compared the adherence of RBCs to P-selectin, to BSA, or to nonbinding Siglec-6 or mutated Siglec-7 chimeras, which share the Ig-Fc domain with the P-selectin construct. The adherence of nonsickle cells to P-selectin is a significant 46% greater than to BSA (146%±16% compared to 100%±0%; P=0.031) and a nearly significant 41% greater than to Siglec-6 or mutated Siglec-7 (146%±16% compared to 104%±9%; P=0.056). The adherence of sickle cells to P-selectin is 72% greater than to BSA (259%±44% compared to 151%±0%; P=0.030) and 74% greater than to Siglec-6 or mutated Siglec-7 (259%±44% compared to 149%±7%; P=0.017). The presence of 5 mM EDTA reduced the adherence to P-selectin by 35% for nonsickle cells (146%±16% to 95%±11%; P=0.027) and 32% for sickle cells (259%±44% to 177%±18%; P=0.016). These statistically significant and near significant differences provide further evidence that normal and, to a larger measure, sickle erythrocytes adhere to P-selectin. The statistically significant reduction of binding resulting from chelating calcium with EDTA confirms the specificity of P-selectin binding. Regarding the EDTA-resistant binding to P-selectin, we previously have established that P-selectin has 2 binding components, one EDTA sensitive and a second that is only sensitive to high (20 mM) concentrations of EDTA. The second component, which is not inhibited by the calcium chelating effect of EDTA but by its polycarboxylic acid nature, may represent the second anion binding site postulated for P- and L-selectin.

J. Effect of Erythrocyte Sialidase Treatment on Their Adherence to Endothelium and to Immobilized P-selectin

The above findings indicate that normal erythrocytes have a ligand for P-selectin, which is enhanced markedly on sickle cells. The only published precedent for P-selectin binding activity on mature RBCs is on malarial parasitized cells, but the origin and nature of that ligand was incompletely characterized and appears to be malarial in origin. Ligand activity for P-selectin is typically mediated by sialylated, fucosylated, sulfated recognition determinants of membrane glycoproteins and glycolipids.

To assess the importance of erythrocyte membrane sialic acid as a binding determinant for P-selectin, we treated RBCs with sialidase before assaying their static adherence to endothelial monolayers, as has been described. Sialidase treatment of erythrocytes reduced adherence to untreated HUVECs by 47% for nonsickle cells (100%±0% to 53%±13%; P=0.018) and by 36% for sickle cells (297%±0% to 191%±67%; P=0.106), Sialidase treatment of RBCs significantly reduced adherence to thrombin-treated HUVECs by 81% for nonsickle (360%±125% to 68%±15%; P=0.047) and 63% for sickle cells (766%±168% to 282%±133%; P=0.044). These data provide a partial characterization of a novel erythrocyte P-selectin ligand that uses sialic acid as a recognition determinant.

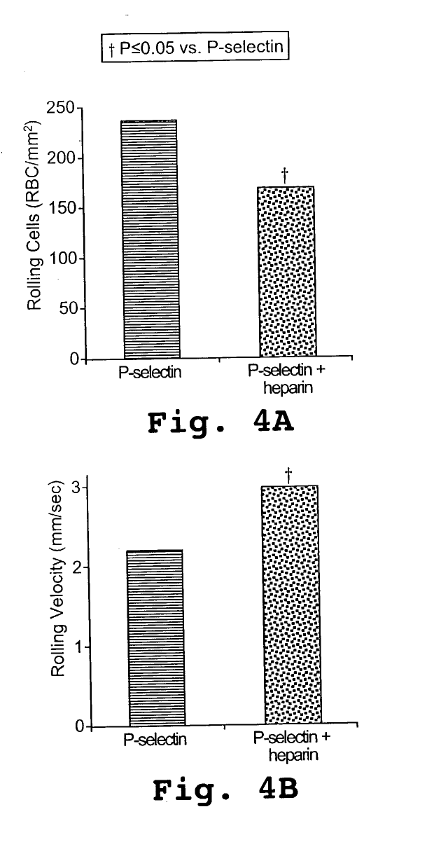

To further explore the importance of sialic acid to the erythrocyte-binding determinant for P-selectin we treated RBCs with sialidase before assaying their nonstatic adherence to P-selectin and to control Siglec-6. The results shown in FIG. 4B demonstrate that treatment of nonsickle cells with sialidase has no significant effect on their adherence to Siglec-6 (110%±11% to 70%±13%; P=0.089) or to P-selectin (134%±14% to 88%±11%; P=0.066). Treatment of sickle cells with sialidase also had no significant effect on their adherence to Siglec-6 (179%±7% to 157%±18%; P=0.089) but a significant 33% reduction in their adherence to P-selectin (273%±28% to 182%±17%; P=0.004). The finding that sialidase causes a statistically significant reduction in the adherence of sickle cells to P-selectin is consistent with the sialidase effects on sickle cell binding to thrombin-treated HUVECs described above. These results further support the partial characterization of a sickle cell P-selectin ligand, which uses sialic acid as a recognition determinant.

To confirm in our system the canonical requirement of sialic acid for P-selectin binding, we compared the effects of treating erythrocytes with sialidase and endothelial cells with mAb 9E1 singly and in combination. We observed that adherence to thrombin-treated endothelial cells was inhibited to a similar degree by sialidase, mAb 9E1, and their combination for nonsickle (17%-24%) and for sickle cells (29%-38%). The lack of additive effect is consistent with the participation of sialic acid and P-selectin in the same molecular interaction and with the adhesion of erythrocytes to P-selectin requiring a sialidase recognition determinant.

Taken together, our data indicate a novel mechanism for sickle cell adherence to thrombin-treated endothelial cells via P-selectin. The partially characterized P-selectin ligand contains sialic acid and is the first reported selectin ligand activity on circulating erythrocytes that are not infected by malaria. The potential importance of P-selectin in sickle cell vaso-occlusion was implicit in the suggestion that sickle cell adhesion may resemble the process of leukocyte adherence. Erythrocyte adhesion to P-selectin also suggests possible molecular mechanisms for the adherence of activated platelets to sickle cells, cooperative heterocellular interactions in sickle cell vaso-occlusion, and the retention of erythrocytes in red thrombi. The modest adherence that we noted of nonsickle cells to P-selectin does not diminish the importance of sickle cell adherence. Indeed, our finding is consistent with previous reports of a lesser degree of nonsickle RBC adherence to endothelial cells. The binding of sickle cells is much more robust than that of nonsickle cells because multiple adhesion systems are involved. The binding of nonsickle cells is weaker and therefore more susceptible to variation in relation to the background “noise” in adherence. When all of our data are taken together, there is evidence for lower level but significant P-selectin-mediated binding of nonsickle cells. The adherence of nonsickle erythrocytes may have little impact on blood flow in a physiologic setting, where normally deformable RBCs easily maneuver past a potential nidus of occlusion. However, 3 important differences distinguish the pathophysiologic setting of sickle cell disease from normal. First, the likely activated condition of endothelial cells in sickle cell disease results in the expression of additional adhesion molecules that presumably strengthen low affinity P-selectin-mediated adherence. Second, the delay in microvascular transit time imposed on circulating sickle cells by the adherent nidus of highly adhesive cells promotes deoxygenation and polymerization of hemoglobin S to generate poorly deformable, reversibly sickled cells. Third, these reversibly sickled cells and the inherently poorly deformable, irreversibly sickled cells reach an impasse behind the adherent nidus to complete the vaso-occlusive process. These conditions that contribute to vaso-occlusion in sickle cell disease are not extant in the normal circulation.

In our studies, P-selectin had significant effects both on static adhesion against the force of gravity orthogonal to the cell surface and on nonstatic adhesion against the tangential shear forces in a rotatory adhesion assay. Others have elucidated the potential enunciated importance of both static and flow adherence studies to sickle cell vaso-occlusion. Based on the flow adhesion models of Springer and coworkers for leukocytes and of Ho and colleagues for Plasmodium falciparum-infected erythrocytes, in which tethering and rolling adhesion is mediated by P-selectin and firm adherence is effected by higher affinity adhesion molecules, it is tempting to predict a greater role for P-selectin in flow than in static adherence. Experiments comparing the effects of P-selectin in assays of the flow and static adherence are described below.

The finding of only partial inhibition of sickle and normal erythrocytes adherence to thrombin-activated HUVECs using blocking P-selectin mAbs with or without sLeX indicates the presence of P-selectin-independent mechanisms of activated adhesion. This is further supported by the partial inhibition of erythrocyte adherence to recombinant P-selectin in the presence of EDTA or with prior sialidase treatment of the RBCs. Possible alternative mechanisms of sickle cell adhesion to thrombin-activated endothelial cells include adherence to the redistributed endothelial integrins or exposed matrix proteins, the use of the putative second ligand binding site of endothelial P-selectin, and the adhesion of endothelial P-selectin to sulfatide in erythrocyte membranes. These sulfated glycolipids have ligand activity for P-selectin and bind the matrix proteins vWF, laminin, and TSP. The sulfated lipid purified by Hillery and colleagues from sickle cell membranes binds TSP and laminin, is resistant to sialidase treatment, and may comprise the sialidase-resistant component of erythrocyte P-selectin ligand activity that we identified.

Detailed understandings of hemoglobin S polymerization notwithstanding, the factors that initiate painful vaso-occlusion in sickle cell disease have not been identified. In this regard, the novel adhesion mechanism we have discovered involves 2 temporal variations with the potential to influence adhesion and occlusion. The expression of P-selectin on endothelial cell surfaces in response to conditions active in sickle cell disease suggests a possible role for such variations in endothelial adhesivity as a determinant of painful vaso-occlusion. Another possible influence on the seemingly random vaso-occlusive events of sickle cell disease could stem from fluctuations in the presentation of P-selectin ligand on sickle RBCs. We have demonstrated that, as with other P-selectin ligands, sialic acid is an important recognition determinant. The primary ligand for P-selectin is P-selectin glycoprotein ligand-1 (PSGL-1), and the precise molecular interactions between P-selectin and this counterreceptor and with the recognition determinant sLeX have recently been solved. We found no evidence of PSGL-1 on sickle RBCs using mAb 2PH1, which is specific for PSGL-1, or KPL1, which is specific for tyrosine sulfated PSGL-1 (both from BD Pharmingen) in flow cytometry (data not shown). We did, however, detect a flow cytometry signal from a fraction of sickle cells using the sLeX mAb HECA-452 (BD Pharmingen; data not shown). The intensity of this sLeX signal varied among patients and over time. The evidence for the presence of sLeX on sickle cell membranes suggests a second possible temporal determinant derived from P-selectin-dependent adhesion. For instance, Lewis RBC antigens are not synthesized by erythrocytes, but consist of glycolipids incorporated into erythrocyte membranes from the plasma into which they are secreted by intestinal epithelial cells. Fluctuations in the synthesis, constitution, plasma concentrations, and membrane incorporation of sLeX and other P-selectin recognition determinants may influence the acquisition of P-selectin ligands by circulating erythrocytes. Such variations could contribute to the variability in sickle cell adhesivity and occurrence of pain. Alternatively, P-selectin ligand on sickle cells, as with certain other adhesion molecules on RBC membranes, may be residual from less mature stages of erythroid development, in which case fluctuations in rates of reticulocytosis may contribute to variations in the adhesivity of sickle cells and the occurrence of pain. We found in flow cytometry experiments that recombinant P-selectin binds only to subpopulations of sickle and nonsickle erythrocytes (data not shown), a binding pattern consistent with either of these 2 mechanisms of ligand presentation.

The complexity of sickle cell adhesion mechanisms will most certainly deepen as the molecular nature of these interactions is defined. For instance, in our preliminary attempts to define the P-selectin ligand we have pretreated erythrocytes with 0.02% trypsin. This reduced substantially the adherence of sickle and nonsickle cells to untreated and thrombin-treated endothelial cells but did not reduce significantly their adhesion to immobilized P-selectin (data not shown). These results suggest that the erythroid P-selectin ligand is probably not a glycoprotein, but that proteolysis of erythrocyte membranes reduces RBC adherence to endothelial cell adhesion molecules other than P-selectin.

Our results suggest that P-selectin be considered as a candidate molecule for new therapeutic approaches for the painful vaso-occlusion of sickle cell disease. New therapeutic strategies include the use of antagonists of endothelial cell activation and inhibitors of P-selectin-ligand interactions, the latter of which includes heparin.

### EXAMPLE 2

Heparin Inhibits the Flow Adhesion of Sickle Red Blood Cells to P-selectin

A. Preparation of Erythrocytes

Blood samples obtained from subjects with sickle cell disease and from healthy control subjects, as approved by the Committee on Human Research at the University of California-San Francisco (UCSF), were drawn into citrate. The buffy coat was removed after the initial centrifugation and after each of 3 subsequent washes of the remaining erythrocytes in phosphate buffered saline (PBS) and one wash in HAH buffer (Hanks balanced salt solution [HBSS; UCSF Cell Culture Facility, San Francisco, Calif.], 1% bovine serum albumin [BSA, Fraction V; Sigma, St Louis, Mo.], 50 mM HEPES [N-2-hydroxyethylpiperazine-N′-2-ethanesulfonic acid; Sigma], pH 7.40). Erythrocytes to be used in flow adhesion studies were suspended to a 0.5% hematocrit. To assess for leukocyte contamination, the erythrocyte preparation was exposed to 10 μg/mL rhodamine 6G, which stains leukocytes but not erythrocytes, and washed 3 times in HAH. No leukocytes were detected in the erythrocyte suspensions prepared using the above protocol.

B. Cell Culture

Human umbilical vein endothelial cells (HUVECs; Clonetics, San Diego, Calif.) were grown in endothelial growth medium (EGM; Clonetics) at 37° C. in 5% CO2 on gelatin-coated glass slides. HUVECs used for the adhesion experiment were no more than third passage and 90% to 95% confluent. To rid HUVECs of the heparin and fetal bovine serum, before treating the cells or assaying for adhesivity, we washed the monolayers 3 times with PBS and once with HAH.

C. Preparation of Immobilized Adhesion Molecules

For studies of adherence to immobilized adhesion protein, 400 ng BSA, recombinant human Siglec-6-Ig chimera (hereafter referred to as recombinant Siglec), or recombinant human P-selectin-Ig chimera (hereafter referred to as recombinant P-selectin) in 10 mM carbonate buffer were applied to slides overnight. Slides were washed with carbonate buffer 3 times, then blocked with 0.5% BSA in HBSS (UCSF Cell Culture Facility).

D. Parallel Plate Flow Adhesion

Because of the vital importance of tethering and rolling adhesion to overall cell adhesion, we elected to measure the parameters of these early adhesive events rather than firm adhesion in our studies of P-selectin. Parallel plate flow adhesion experiments were performed as previously described. Slides containing HUVECs or immobilized protein were attached to the base of a CytoShear parallel plate chamber (CytoDyne, San Diego, Calif.). All experiments were performed with cells and reagents in HAH. A shear stress of 1 dyne/cm², which is estimated as that of normal postcapillary venular flow, was maintained using a Sage syringe pump (Orion, Beverly, Mass.). Temperature was maintained at 37° C. using Heating Tape (Fisher, Pittsburgh, Pa.) and a Temperature Controller (Cole-Parmer, Vernon Hills, Ill.).

Rolling cells were defined as those sickle cells that were in the same microscopic focal plane as the immobile surface or endothelial monolayer and moving at a distinctly slower velocity than the bulk flow. Those cells determined to be rolling were in contact with the substrata throughout their entire transit through the field of observation, which excludes those cells in brief transient contact with the substrate. Data on rolling adherence are expressed as the mean numbers of rolling red blood cells (cells/mm²) and the mean rolling velocity of these cells (mm/s) from multiple experiments; error is expressed as SEM. The microscopic field in which adherent cells were measured has an area of 0.15 mm² and a volume of 0.038 mm³. In serial assessments of adhesion under different experimental conditions, only rare firmly adherent sickle cells were encountered. In such instances, a different microscopic field was selected randomly for determining rolling adhesion. The velocity of rolling cells was determined by the time required for a cell to roll across the 0.5-mm field (0.5 mm/s). Firmly adherent cells were defined as those cells whose location, under flow conditions, do not change over a 1-second (30-frame) observation time. Data on firm adhesion are expressed as the mean numbers of firmly adherent red blood cells in the area described above. Statistical significance was determined using the Student t test.

E. Endothelial Cell Activation and Adhesion-blocking Treatments

In some experiments, HUVECs were treated with 0.1 U/mL thrombin by exposure for 5 minutes in the parallel plate chamber. For blocking experiments, a 1:200 dilution of adhesion-blocking anti-P-selectin monoclonal antibody (mAb) 9E1 (R&D Systems, Minneapolis, Minn.), a 1:200 dilution of nonblocking control anti-P-selectin mAb AC1.2 (BD Pharmingen, San Diego, Calif.), 400 μg/mL laboratory-grade unfractionated porcine intestine heparin (Sigma), or 400 μg/mL laboratory-grade low-molecular-weight porcine intestinal heparin (Sigma) was infused into the parallel plate chamber over thrombin-treated HUVECs or immobilized recombinant P-selectin. This concentration of heparin is similar to published concentrations used in adhesion-blocking experiments and is equivalent to 50 U/mL. We also compared the antiadhesion effects of laboratory-grade (Sigma) and clinical-grade (Elkins-Sinn, Cherry Hill, N.J.) unfractionated heparins at concentrations encompassing 4 orders of magnitude.

These interventions and the measurements of flow adhesion were made serially, in the following order: control adhesion to unstimulated endothelial monolayers, thrombin infusion, adhesion to activated endothelial monolayers, adhesion-blocking agent (mAb and/or heparin), and adhesion to blocked monolayers. In the case of immobilized.adhesion proteins, thrombin infusion was not used.

F. The Flow Adhesion of Sickle Cells is Mediated by P-selectin in vitro

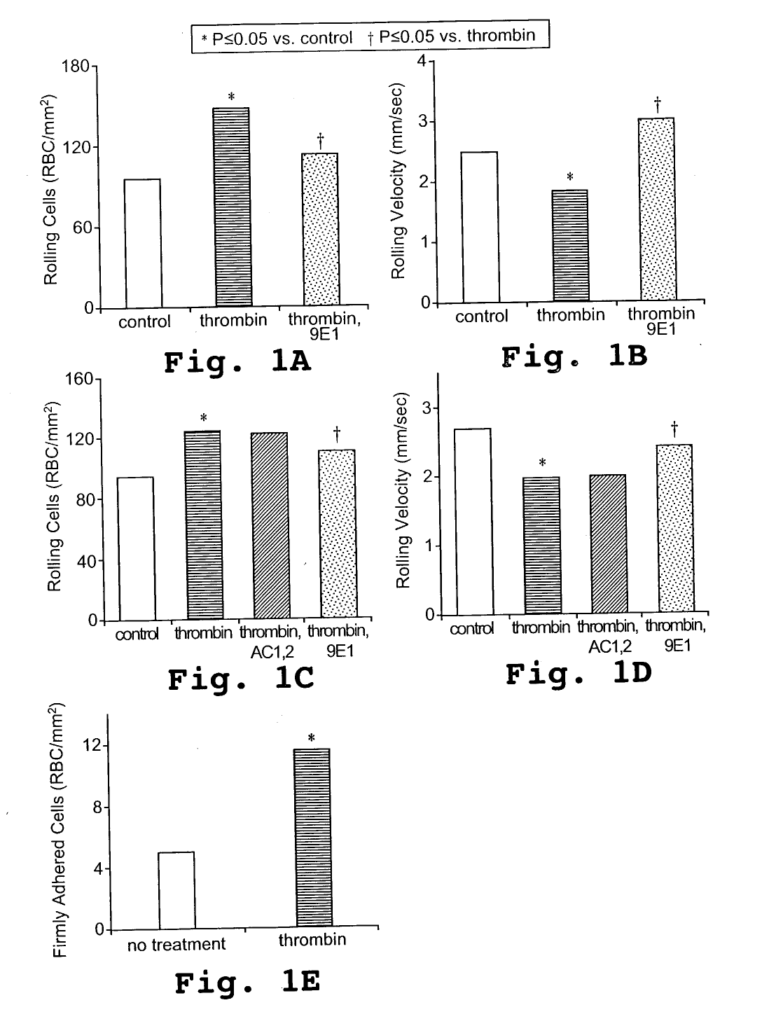

To test whether P-selectin has a role in the adhesion of sickle cells to thrombin-treated endothelium under flow conditions in vitro, we used a parallel plate chamber under ambient oxygen conditions. Under these conditions, the polymerization of hemoglobin S is not induced,:and the sickle cells retain their antecedent shape. In 10 replicate experiments, we found that the flow adhesion of sickle erythrocytes was indeed increased after thrombin treatment of the HUVECs (FIGS. 1A-1B). All adherent cells had the shape of normal biconcave discs. Treatment of HUVECs with 0.1 U/mL thrombin for 5 minutes increases the number of rolling sickle cells by 54% (P<0.001, n=10; FIG. 1A) and decreases their rolling velocities by 26% (P<0.001, n=10; FIG. 1B). P-selectin antibody treatment of endothelial cells decreased thrombin-enhanced rolling adhesion in vitro (FIGS. 1A-1B). P-selectin mAb 9E1 reduces thrombin-enhanced adhesion of rolling cells by 68% (P=0.002, n=10; FIG. 1A) and increases their rolling velocities by 72% (P<0.001, n=10; FIG. 1B). The nonblocking P-selectin mAb AC1.2 (isotype matched to 9E1) does not significantly affect the thrombin-enhanced number of rolling sickle cells (FIG. 1C) or their rolling velocities (FIG. 1D). Compared with mAb AC1.2, mAb 9E1 reduces the thrombin effect on number of rolling cells (P=0.030, n=3) and the rolling velocity (P=0.007). These results indicate that the thrombin-enhanced component of rolling adhesion of sickle cells to endothelial cells primarily involves P-selectin.

Firm adhesion of sickle cells to HUVECs was also enhanced by thrombin treatment. Compared with the 95.4 sickle cells rolling per millimeters squared on untreated HUVECs, only 5.0 sickle cells were firmly adhered (n=10). Thrombin treatment of HUVECs increases the number of firmly adhered cells by 130% (P<0.001, n=10; FIG. 1E). Treatment with mAb 9E1 causes no statistically significant change in the number of cells firmly adhered to thrombin-treated HUVECs. These results indicate that thrombin-enhanced firm adhesion involves molecular pathways in addition to P-selectin.

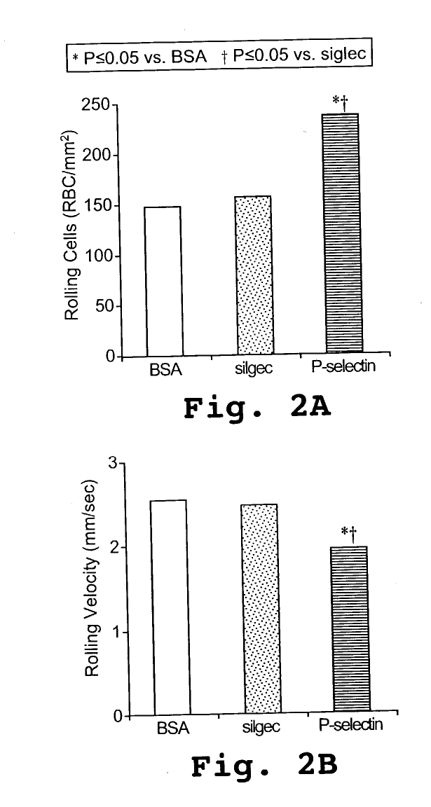

We independently verified that sickle cell flow adhesion involves P-selectin in studies with immobilized recombinant P-selectin in vitro (FIG. 2). Immobilized BSA supported the rolling of 148 sickle cells/mm2 (1.17% of the total sickle cells) at a velocity of 2.54 mm/s, as well as 0.81 firmly adhered sickle cells/mm2. This level of adhesion to BSA reflects the innate adhesivity of proadhesive sickle cells. Immobilized recombinant Siglec (which does not recognize ligands on erythrocytes and has a human Ig-Fc tail) gives similar results, supporting the rolling of 157 sickle cells/mm2 (1.24%) at a velocity of 2.48 mm/s. Immobilized recombinant P-selectin supports 50% more rolling sickle cells than does the immobilized recombinant Siglec control (P=0.005, n=5) and 59% more than does the immobilized BSA control (P=0.009, n=5; FIG. 2A). The rolling velocity of sickle cells on recombinant P-selectin is 21% slower than on immobilized recombinant Siglec (P=0.004, n=5) and 23% slower than on BSA (P=0.026, n=5; FIG. 2B). Sickle erythrocyte rolling on immobilized recombinant Siglec is not significantly different than erythrocyte rolling on immobilized BSA. The number of rolling nonsickle erythrocytes increased by only 14.8% on immobilized recombinant P-selectin compared with BSA (P=0.019, n=4); there was no statistically significant change in their velocities. The firm adhesion to immobilized recombinant P-selectin compared with BSA was not significantly different for either sickle or nonsickle erythrocytes. This lesser level of firm adherence on recombinant P-selectin, compared with that observed on thrombin-treated HUVECs, again reflects the participation of endothelial cell adhesion mechanisms other than P-selectin. These results confirm that P-selectin can mediate specifically the rolling adhesion of sickle red cells.

G. Heparin Inhibits the Thrombin-enhanced Flow Adhesion of Sickle Erythrocytes to Endothelial Cell P-selectin in vitro

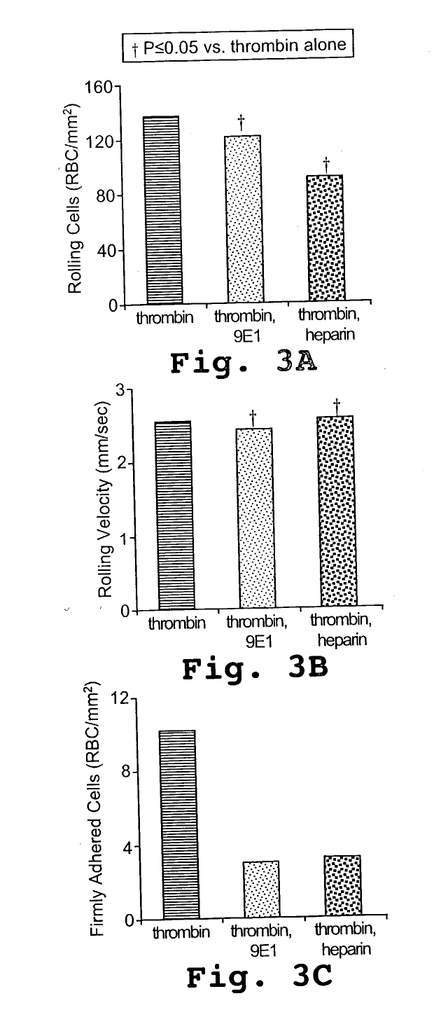

In 6 replicate experiments, we tested the efficacy of heparin in blocking the thrombin-enhanced component of rolling adhesion of sickle cells to HUVECs in vitro (FIG. 3). Unfractionated laboratory-grade heparin reduces the number of sickle cells rolling on thrombin-treated HUVECs by 93% (P=0.004; FIG. 3A) and increases the velocity of these cells by 113% (P<0.001; FIG. 3B). There is no significant difference between inhibition by mAb 9E1 and unfractionated heparin for either the number of rolling cells or their rolling velocities, which is consistent with.each eliciting its inhibitory effect on P-selectin. We also found that the combination of anti-P-selectin mAb 9E1 and heparin is not more effective than either agent alone (data not shown). These results indicate that heparin is blocking primarily the P-selectin-mediated in vitro flow adhesion and is not blocking P-selectin-independent mechanisms.

We also found that unfractionated heparin reduces the thrombin-enhanced number of rolling cells by 110% and increases their velocities by 78%, but that low-molecular-weight heparin reduces the thrombin-enhanced number of rolling cells by only 58% and increases their velocities by just 10%. This result is consistent with unfractionated heparin having a greater effect than low-molecular-weight heparin on the flow adhesion of sickle red cells to P-selectin, as also is true for the binding of P-selectin to immobilized sLeX.

The low level of firm adherence of sickle cells to thrombin-treated HUVECs is reduced by either P-selectin blocking mAb 9E1 (n=3; P=0.058) or unfractionated heparin (n=3; P=0.057; FIG. 3C). The similar levels of inhibition suggest that both agents are blocking P-selectin.

We also directly verified that sickle cell adhesion to P-selectin is inhibited by unfractionated laboratory-grade heparin by testing for in vitro flow adhesion to immobilized recombinant P-selectin (FIG. 4). In 3 replicate experiments, we tested the efficacy of heparin in blocking the flow adhesion of sickle cell to immobilized recombinant P-selectin in vitro (FIG. 4). Unfractionated heparin reduces the number of rolling cells by 29% (P=0.051; FIG. 4A) and increased the velocity by 34% (P=0.045; FIG. 4B). As with rolling adhesion to thrombin-activated endothelial cells, we found that the combination of unfractionated heparin and anti-P-selectin mAb 9E1 decreased the number and increased the velocity of rolling cells on immobilized recombinant P-selectin to a similar extent (by 27% and by 57% respectively). These results are similar to those with heparin alone, which decreased the number of rolling cells by 35% and increased their rolling velocity by 30%, and to those with anti-P-selectin mAb 9E1 alone, which decreased the number of rolling cells by 39% and increased the rolling velocity by 58%. The low level of firm adherence of sickle cells to recombinant P-selectin is not significantly altered by mAb 9E1 or unfractionated heparin. Neither the rolling nor the firm adhesion of nonsickle erythrocytes on recombinant P-selectin is significantly affected by mAb 9E1 or unfractionated heparin. Overall, these findings indicate that the main role of P-selectin is to mediate the initial tethering and rolling adhesion of sickle cells, rather than their firm adhesion.

We also compared the effects of laboratory-grade unfractionated and low-molecular-weight heparins to immobilized recombinant P-selectin. We found that unfractionated heparin reduces the number of rolling cells by 27% and increases their velocity by 24%, whereas low-molecular-weight heparin reduces the number of rolling cells by only 6% and increases their velocity by just 6%. These results confirm that heparin can block P-selectin-mediated in vitro flow and are consistent with unfractionated heparin having a greater effect than low-molecular-weight heparin on the flow adhesion of sickle red cells to recombinant P-selectin. However, in this experiment we tested only one of the many types of low-molecular-weight heparins currently available.

The background adherence seen in all experiments could represent other biologically relevant adhesion systems that are not affected by thrombin activation. The background adhesion detected in studies using pure molecules (including BSA), suggests that much of the baseline adhesion is due to nonspecific stickiness of sickle erythrocytes. Regardless, the thrombin-dependent component with HUVECs is clearly shown to be due to P-selectin and this is confirmed by the studies with recombinant P-selectin. Likewise, the inhibitory effects of heparin can be explained by P-selectin blockade.

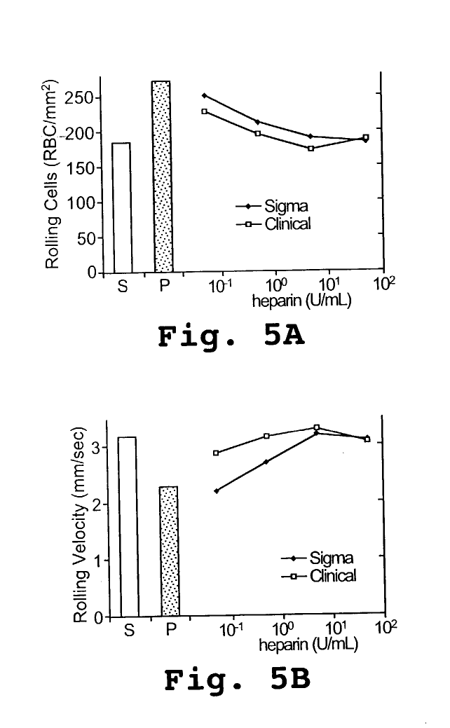

In the above studies, we had used high concentrations of unfractionated laboratory-grade heparin, similar to those described in the literature. To assess the clinical relevance for heparin therapy, we also compared the capacity of several concentrations of clinical-grade (Elkins-Sinn) and laboratory-grade (Sigma) unfractionated heparin to block the adhesion of sickle erythrocytes to immobilized recombinant P-selectin. Both types of heparin reduce the adhesion of sickle cells to immobilized recombinant P-selectin (FIG. 5). Both also cause a decrease in the number of rolling cells and an increase in their rolling velocities at all concentrations tested, including concentrations attained in the plasma during clinical administration (ie, 0.2-0.4 U/mL)0.37 At 5 U/mL to 50 U/mL, rolling adhesion was approximately the same as that of the basal adhesion to BSA. These results indicate a similarity in the effects of laboratory and clinical grades of heparin with regard to the inhibition of P-selectin-dependent sickle cell-endothelial cell adhesion and indicate their therapeutic relevance.

H. Discussion

P-selectin mediates the flow adhesion of sickle erythrocytes to thrombin-activated endothelial cells in vitro. Furthermore, this thrombin-enhanced adhesion can be inhibited by antibodies to P-selectin or by unfractionated heparin. Thrombin causes a rapid increase in endothelial cell adhesivity for sickle erythrocytes. Within 5 minutes of thrombin stimulation, the adhesion of sickle cells to endothelial cells markedly increases.

Upon thrombin stimulation, P-selectin in Weibel-Palade bodies rapidly translocates and is rapidly expressed on the luminal surface of the endothelial cell. Previously, we have shown that P-selectin mediates thrombin-enhanced static adhesion of sickle erythrocytes to endothelial cells in vitro. This static adhesion is mediated by an unknown ligand on sickle erythrocytes. The susceptibility of this adhesion to sialidase treatment of erythrocytes indicates that the unknown ligand bears critical sialic acid residues. The lack of inhibition by trypsin treatment of erythrocytes suggests that the unknown ligand may not be a protein. Furthermore, the variable detection of sLeX on some samples of sickle cells also suggests that in some cases a sLeX moiety may have a role.

Our results show that the number of rolling sickle cells increases and that their velocity decreases as a result of thrombin treatment of HUVECs. Studies with blocking and nonblocking anti-P-selectin mAb confirm that this enhancement is P-selectin-mediated under flow conditions.

Our findings that both unfractionated heparin and anti-P-selectin mAb 9E1 reduce thrombin-enhanced rolling adhesion of sickle cells to HUVECs and adhesion to immobilized recombinant P-selectin indicate that heparin is acting on P-selectin in this capacity. Low-molecular-weight heparin also. causes a decrease in P-select-independent sickle cell adhesion to HUVECs. The inhibition of sickle cell adhesion by low-molecular-weight heparin that we report here is based on the use of only one of the many types of low-molecular-weight heparins currently available. Given the generally more favorable pharmacology and toxicity profiles of low-molecular-weight heparin, these preparations are contemplated for use in certain embodiments of the invention.

Our data are consistent with published findings that adhesive mechanisms other than P-selectin are involved in sickle cell adhesion to activated endothelium. In the current model of neutrophil adhesion to endothelium, selectins mediate the initial steps as they are well suited for tethering rapidly flowing cells and slowing them down as they associate with the vascular wall. According to this model, activated endothelium expressing intercellular adhesion molecule-1 (ICAM-1) can then mediate firm adhesion by binding neutrophil 2 integrins. We postulate that, in a manner similar to that seen for neutrophil adhesion, P-selectin may play a role in the tethering and rolling adhesion of sickle cells (FIGS. 1 and 2). As with neutrophils, integrins may then mediate the firm adhesion of rolling sickle erythrocytes. The integrin (α₄β₄ is expressed on sickle reticulocytes and can mediate adhesion to endothelial cells, possibly via endothelial VCAM-4. The endothelial integrin, αᵥβ₃, also mediates sickle cell adhesion to endothelial cells. Other β₁ and β₃ integrins may also fulfill this role. A more thorough investigation of cooperation between multiple adhesion mechanisms will be required to confirm our prediction that, like neutrophils, sickle erythrocytes too utilize a multistep model of adhesion to initiate vascular occlusion.

In addition to the above-mentioned pathways, other sickle cell-endothelial cell adhesion mechanisms have been described. Recently, integrin-associated protein (CD47) has been demonstrated to activate adhesivity in sickle reticulocyte as well as mediate adhesion to TSP. Whereas CD36 (GPIV) has been implicated in earlier studies to mediate adhesion of sickle cells, the presence or absence of CD36 on sickle reticulocytes and erythrocytes does not affect the clinical course. Band 3 protein also can mediate sickle cell-endothelial cell adhesion.

Heparin has traditionally been used as an anticoagulant. Its effects against P-selectin-mediated tumor cell adhesion and inflammation also have been described. The TSP-mediated adhesion of sickle cells to endothelial cells and to the mesocecal vasculature of rats can be blocked by heparin or heparan sulfate. We found that heparin also can inhibit adhesion in the absence of plasma or added soluble ligands (FIGS. 3 and 4). These effects of heparin gain perspective from the suggestion that heparin therapy is beneficial as prophylaxis for patients with recurrent painful sickle cell crises.

In addition to its anticoagulant, TSP-blocking, and selectin-blocking effects, heparin has numerous other actions. It is known to bind to and inhibit the action of IL-8 and other chemokines, which, in effect, reduces endothelial integrin activation. It also competes with hyaluronate binding of CD44. Damage by reactive oxygen species is reduced by heparin; this may indirectly affect the expression of endothelial adhesion molecules.

Several approaches to interfering with P-selectin-mediated adhesion are under study or development. Experiences with blocking selectin-ligand binding using antibodies against P-selectin such as 9E1, recombinant selectin-Ig chimeras, oligosaccharide components of natural selectin ligands such as sLex or the amino-terminal domain of PSGL-1, or oligopeptides derived from P-selectin sequences have been reviewed. A powerful new strategy for developing polyvalent synthetic selectin-binding ligands that are orders of magnitude more potent than their monosaccharide components has capitalized on the much greater binding strength of polyvalent cell surface glycoprotein structures compared with their monomeric oligosaccharide constituents. Another suggested strategy relies upon small synthetic oligosaccharides, glycoconjugates, glycomimics, and unnatural substrates to modulate metabolically the biosynthesis, processing, assembly, or structure of adhesive glycoconjugates on cell surfaces.

In addition to these elegant new molecular strategies, heparin has much to recommend it as an agent for inhibiting P-selectin-mediated sickle cell binding. The extensive clinical experience with its use, side effects, and dosing make it a compelling candidate for clinical trials of the prevention of painful vascular occlusion in sickle cell disease. The potential role of P-selectin as the initial adhesive process that initiates vascular occlusion suggests that heparin therapy would be more effective as prophylaxis than as treatment for established pain crises. We found that clinical-grade unfractionated heparin can inhibit partially P-selectin-dependent adhesion of sickle cells at concentrations attained in the plasma during clinical use (FIG. 5). Significant concerns with prolonged heparin therapy are abnormal bleeding, heparin-induced thrombocytopenia, and inconvenience of administration. While the risk of bleeding is to some degree circumvented by the substantial clinical experience with heparin dosing, the issues of thrombocytopenia and convenience of administration remain. The use of subcutaneous low-molecular-weight heparin would permit more convenient administration and is associated with a lower incidence of heparin-induced thrombocytopenia, but its potential for preventing vascular occlusion may be diminished by its limited efficacy in blocking P-selectin binding, peculiar inability to increase tissue factor pathway inhibitor levels in sickle cell disease, and requirement for parenteral administration. The discomfort and inconvenience associated with long-term parenteral therapies lessens enthusiasm for the prophylactic administration of heparin. Despite assertions of the lack of absorption of orally administered heparin because of its large molecular weight, strong negative charge, and hydrophilicity, there are published reports that unfractionated heparin administered orally to laboratory animals is absorbed, binds avidly to the endothelium, and has antithrombotic activity. One particular formulation of heparin with an agent that promotes its oral absorption has been reported to have antithrombotic activity and possibly to be associated with a lower incidence of heparin-associated thrombocytopenia. These issues taken together with the findings we have presented herein indicate that the time has come for a clinical trial of the of unfractionated heparin, administered by the oral route, for the prevention of painful vascular occlusion in sickle cell disease.

The use of heparin to inhibit the adhesive events important to sickle cell vascular occlusion may affect also multicellular events, such as those described for carcinoma emboli. There is evidence that both platelets and leukocytes facilitate carcinoma cell metastasis, that both P- and L-selectin participate in the process, and that heparin can inhibit both selectin molecules. Regarding multicellular interactions in sickle cell vascular occlusion, it has been reported that the addition of platelets enhances the static adhesion of sickle cells to the vascular endothelium in vitro and that in mouse models of sickle cell disease, leukocyte adhesion may precede that of sickle erythrocytes. It also has been reported that in sickle cell disease platelets express P-selectin and adhere to sickle red cells in circulating clumps. Additionally, neutrophils are found in increased numbers in sickle cell disease, are often activated, and have been reported to bind to both endothelial cells and sickle erythrocytes. These findings are consistent with multicellular adhesion involving endothelial cells, sickle cells, platelets, and neutrophils in vascular occlusion, with a potential role for P- and/or L-selectin in such processes. These proposed interactions provide further support for a therapeutic trial of heparin in sickle cell disease.

### EXAMPLE 3

Generation of LMWH

Rationally designed LMWHs were generated through the controlled cleavage of porcine intestinal mucosa heparin with a mixture of heparinases. Briefly, to 1 g of porcine intestinal mucosa in 50 ml of 50 mM calcium acetate buffer, pH 6.7, 0.1 molar equivalent of a heparinase mixture was added, and the solution was maintained at 37° C. for 4-8 h. After precipitation of the enzyme, the supernatant was loaded onto a 1-m long, 10-cm diameter P10 size exclusion column. Saccharide fragments were eluted by using a running buffer of 100 mM ammonium bicarbonate, pH 9.0. The eluent was tracked by UV absorption at 232 nm, and 3-ml fractions were collected after the initial void volume. The fractions yielding positive UV absorption at 232 nm were collected and pooled. The sample was lyophilized to remove ammonium bicarbonate and redissolved in ultrapure water.

It is claimed:

1. A method of preventing pain in a sickle cell patient, comprising
orally administering to the patient, an amount of heparin effective on oral administration to inhibit binding of the patient's sickle erythrocytes to P-selectin on the patient's vascular endothelium, as evidenced by one or more of the group consisting of enhanced microvascular blood flow in conjunctivae of the patient relative to microvascular blood flow prior to treatment, enhanced vascular endothelial well-being in the patient relative to vascular endothelial well-being prior to treatment, and prevention or reduced frequency of pain crises in the patient relative to pain crises prior to treatment,
by said administering, inhibiting the adhesion of sickle erythrocytes to vascular endothelium in the patient, thereby to prevent patient pain associated with vascular occlusion.

2. The method of claim 1, wherein the inhibition is evidenced by enhanced microvascular blood flow in conjunctivae of the patient relative to microvascular blood flow prior to treatment, and where the blood flow is monitored with computer-assisted intravital microscopy or Laser-Doppler velocimetry in vivo.

3. The method of claim 1, wherein the inhibition is evidenced by enhanced vascular endothelial well-being in the patient relative to vascular endothelial well-being prior to treatment as determined by one or more surrogate markers of vascular endothelial well-being, where the surrogate marker is selected from the group consisting of soluble P-selectin (sP-sel), vascular endothelial cell adhesion molecule-1 (sVCAM-1), tumor necrosis factor-a (TNFa), Interleukin-1b (IL-1b), IL-6, IL-8, IL-10, a2-macroglobulin, C-reactive protein (CRP), high sensitivity CRP, soluble interleukin-2 receptor (slL-2R), substance P, endothelin-1, circulating endothelial cells (CEC), microparticles (MP) from the plasma membranes of endothelial cells, MP from monocytes, platelets, and sickle RBC.

4. The method of claim 1, wherein the inhibition is evidenced by prevention or reduction in frequency of pain crises in the patient relative to pain crises prior to treatment.

5. The method of claim 1, wherein the heparin administered is a non-anticoagulant form of heparin formed by desulfating heparin at the 2-O position of uronic acid residues and the 3-O position of glucosamine residues of heparin.

6. The method of claim 1, wherein the heparin administered is unfractionated porcine heparin.

7. The method of claim 1, wherein the heparin administered is produced by treating porcine heparin with a mixture of heparinases, under conditions effective to produce an average molecular weight of heparin between 4 and 6 kilodaltons.

8. The method of claim 1, wherein the heparin administered is complexed with an enhancer compound effective to enhance the uptake of the heparin from the GI tract into the bloodstream.

9. The method of claim 8, wherein said enhancer compound is selected from the group consisting of sodium N-[8-(2-hydroxybenzoyl)amino] caprylate (SNAC), sodium N-[8-(2-hydroxybenzoyl)amino] decanoate (SNAD), Orasomes, Promdas, Locdas, Hydroance, Lipral, Labrasol (caprylocaproyl macrogolglycerides), D-β-tocopheryl polyethylene glycol 1000 succinate (TPGS), DOCA, alginate/poly-L-lysine microparticles, polycarbophil, hydroxypropyl methylcellulose, carbopol 934, sodium salicylate, polyoxyethylene-9-lauryl ether, poly(ethylcyanoacrylate) (PECA), 2-alkoxy-3-alkylamidopropylphosphocholines, dodecylphosphocholine (DPC), and poly(diethyl)methylidenemalonate (DEMM).

10. The method of claim 9, wherein said enhancer compound is Hydroance.

11. The method of claim 1, wherein the heparin administered is in a tablet or capsule designed to increase absorption from the GI tract.

12. The method of claim 1, wherein said administering is carried out on a daily basis, at a daily dose of between about 40 mgs to about 2700 mgs heparin.

13. The method of claim 12, wherein said daily dose is between about 50 mgs to about 600 mgs heparin.

14. A composition for use in preventing pain in a sickle-cell patient, comprising heparin contained in a solid or capsule form suitable for oral administration, at a total dose of between about 50 to 500 mg heparin.

15. The composition of claim 14, wherein the heparin is a non-anticoagulant form of heparin formed by desulfating heparin at the 2-O position of uronic acid residues and the 3-O position of glucosamine residues of heparin.

16. The composition of claim 14, wherein the heparin is unfractionated porcine heparin.

17. The composition of claim 14, wherein the heparin is produced by treating porcine heparin with a mixture of heparinases, under conditions effective to produce an average molecular weight of heparin between 4 and 6 kilodaltons.

18. The composition of claim 14, wherein the heparin is complexed with an enhancer compound effective to enhance the uptake of the heparin from the GI tract into the bloodstream.

19. The composition of claim 18, wherein said enhancer compound is selected from the group consisting of sodium N-[8-(2-hydroxybenzoyl)amino] caprylate (SNAC), sodium N-[8-(2-hydroxybenzoyl)amino] decanoate (SNAD), Orasomes, Promdas, Locdas, Hydroance, Lipral, Labrasol (caprylocaproyl macrogolglycerides), D-α-tocopheryl polyethylene glycol 1000 succinate (TPGS), DOCA, alginate/poly-L-lysine microparticles, polycarbophil, hydroxypropyl methylcellulose, carbopol 934, sodium salicylate, polyoxyethylene-9-lauryl ether, poly(ethylcyanoacrylate) (PECA), 2-alkoxy-3-alkylamidopropylphosphocholines, dodecylphosphocholine (DPC), and poly(diethyl)methylidenemalonate (DEMM).

20. The composition of claim 19, wherein said enhancer compound is Hydroance.
